SEQUENCE LISTING (45 sequences)

SEQ ID NO: 1 - DNA, 20 bp, Artificial
  Desmin_for
gctatcaggacaacattgcg

SEQ ID NO: 2 - DNA, 20 bp, Artificial
  Desmin_rev
gttgttgctgtgtagcctcg

SEQ ID NO: 3 - DNA, 21 bp, Artificial
  alpha-SMA_for
ctattcaggctgtgctgttcc

SEQ ID NO: 4 - DNA, 20 bp, Artificial
  alpha-SMA_rev
ggacctcttctcgatgctga

SEQ ID NO: 5 - DNA, 20 bp, Artificial
  Vimentin_for
tagcaggacactattggccg

SEQ ID NO: 6 - DNA, 20 bp, Artificial
  Vimentin_rev
ctgttgcaccaagtgtgtgc

SEQ ID NO: 7 - DNA, 21 bp, Artificial
  VE-cadherin_for
aggctgaatacaagatcgtgg

SEQ ID NO: 8 - DNA, 21 bp, Artificial
  VE-cadherin_rev
ggtctgtctcaatggtgaagg

SEQ ID NO: 9 - DNA, 21 bp, Artificial
  PECAM_for
ccacttctgaactccaacagc

SEQ ID NO: 10 - DNA, 19 bp, Artificial
  PECAM_rev
ccaacatgaacaaggcagc

SEQ ID NO: 11 - DNA, 20 bp, Artificial
  Flk-1_for
tctttcggtgtgttgctctg

SEQ ID NO: 12 - DNA, 20 bp, Artificial
  Flk-1_rev
taggcagggagagtccagaa

SEQ ID NO: 13 - DNA, 20 bp, Artificial
  CD16
ccacaactggagttccatcc

SEQ ID NO: 14 - DNA, 22 bp, Artificial
  CD16_rev
ttgttcctccagctatggcacc

SEQ ID NO: 15 - DNA, 20 bp, Artificial
  AFP_for
acaggaggctatgcatcacc

SEQ ID NO: 16 - DNA, 20 bp, Artificial
  AFP_rev
tggacatcttcaccatgtgg

SEQ ID NO: 17 - DNA, 20 bp, Artificial
  TO_for
gctcaaggtgatagctcgga

SEQ ID NO: 18 - DNA, 20 bp, Artificial
  TO_rev
ggaactctgccatctgttcc

SEQ ID NO: 19 - DNA, 21 bp, Artificial
  TAT_for
tccaggagttctgtgaacagc

SEQ ID NO: 20 - DNA, 20 bp, Artificial
  TAT_rev
agtatatggtgcctgcctgc

SEQ ID NO: 21 - DNA, 21 bp, Artificial
  beta actin_for
tggagaagagctatgagctgc

SEQ ID NO: 22 - DNA, 20 bp, Artificial
  beta actin_rev
gatccacatctgctggaagg

SEQ ID NO: 23 - DNA, 20 bp, Artificial
  AFP_for
atcgacctcaccgggaagat

SEQ ID NO: 24 - DNA, 21 bp, Artificial
  AFP_rev
gagttcacagggcttgcttca

SEQ ID NO: 25 - DNA, 25 bp, Artificial
  AFP_t; additionally having reporter dye FAM at the 5′ end and quencher dye TAMRA at the 3′ end
aatgtcggccattccctcaccacag

SEQ ID NO: 26 - DNA, 19 bp, Artificial
  TAT_for
tgacgagtggctgcagtca

SEQ ID NO: 27 - DNA, 23 bp, Artificial
  TAT_rev
tgacctcaatccccatagactca

SEQ ID NO: 28 - DNA, 26 bp, Artificial
  TAT_t; additionally having reporter dye FAM at the 5′ end and quencher dye TAMRA at the 3′ end
tggacagaacatcctcattccgaggc

SEQ ID NO: 29 - DNA, 22 bp, Artificial
  TO_for
ggtgaacgacgactgtcatacc

SEQ ID NO: 30 - DNA, 22 bp, Artificial
  TO_rev
catgagcgtgtcaatgtccata

SEQ ID NO: 31 - DNA, 27 bp, Artificial
  TO_t; additionally having reporter dye FAM at the 5′ end and quencher dye TAMRA at the 3′ end
tacagggaagagcctcgattccaggtc

SEQ ID NO: 32 - DNA, 20 bp, Artificial
  GFP_for
aagcagcacgacttcttcaa

SEQ ID NO: 33 - DNA, 20 bp, Artificial
  GFP_rev
cggccatgatatagacgttg

SEQ ID NO: 34 - DNA, 20 bp, Artificial
  AFP_for
tgctgcaaattacccatgat

SEQ ID NO: 35 - DNA, 20 bp, Artificial
  AFP_rev
aaggttggggtgagttcttg

SEQ ID NO: 36 - DNA, 21 bp, Artificial
  Fxa2_for
agtggatcatggacctcttcc

SEQ ID NO: 37 - DNA, 20 bp, Artificial
  Foxa2_rev
cttccttcagtgccagttgc

SEQ ID NO: 38 - DNA, 21 bp, Artificial
  TAT_for
tccaggagttctgtgaacagc

SEQ ID NO: 39 - DNA, 20 bp, Artificial
  TAT_rev
agtatatggtgcctgcctgc

SEQ ID NO: 40 - DNA, 20 bp, Artificial
  TO_for
gctcaaggtgatagctcgga

SEQ ID NO: 41 - DNA, 20 bp, Artificial
  TO_rev
ggaactctgccatctgttcc

SEQ ID NO: 42 - DNA, 21 bp, Artificial
  G6P_for
tgcattcctgtatggtagtgg

SEQ ID NO: 43 - DNA, 20 bp, Artificial
  G6P_rev
gaatgagagctcttggctgg

SEQ ID NO: 44 - DNA, 20 bp, Artificial
  GAPDH_for
attcaagggcacagtcaagg

SEQ ID NO: 45 - DNA, 20 bp, Artificial
  GAPDH_rev
atcataaacatgggggcatc

(19) United States
(12) Patent Application Publication
(10) Pub. No.: US 2006/0205075 A1
(43) Pub. Date: Sep. 14, 2006

(54) In vitro differentiation and maturation of mouse embryonic stem cells into hepatocytes

(75) Inventors: Norio Nakatsuji, Kyoto-shi (JP); Kentaro Yasuchika, Kyoto-shi (JP); Takamichi Ishii, Kyoto-shi (JP); Toshitaka Hoppo, Kyoto-shi (JP); Iwao Ikai, Kyoto-shi (JP); Tetsuro Hirose, Kyoto-shi (JP); Hideaki Fujii, Kyoto-shi (JP); Hajime Kubo, Kyoto-shi (JP); Naoko Kamo, Kyoto-shi (JP)

Correspondence Address: DARBY & DARBY P.C., P. O. BOX 5257, NEW YORK, NY, 10150-5257, US

(73) Assignee: Kyoto University, Kyoto-shi (JP)

(21) Appl. No.: 11/373,760

(22) Filed: Mar. 9, 2006

Related U.S. Application Data
(60) Provisional application No. 60/660,692, filed on Mar. 10, 2005.

(51) Int. Cl. A61K 35/12; C12N 5/08

(52) U.S. Cl. 435/370; 424/93.7

(57) ABSTRACT

The present invention provides a method for preparing a mature hepatocyte from an embryonic stem cell in vitro, comprising: (a) culturing the embryonic stem cell so as to differentiate into an endodermal cell; (b) isolating the endodermal cell from a population of the differenciated cell; and (c) culturing the isolated endodermal cell in the presence of a Thy 1-positive mesenchymal cell.

### BACKGROUND OF THE INVENTION

1. Field of the Invention

The present invention relates to an in vitro method for producing matured hepatocytes from embryonic stem cells, matured hepatocytes produced thereby, and use thereof.

2. Description of the Related Art

Because of a shortage of donors for liver transplantation, cell transplantation has been explored as a useful bridge or alternative therapy. Hepatocyte transplantation can improve liver function sufficiently to extend the waiting time for liver transplantation [1-4]. However, using this as an effective clinical therapy requires the development of a cell source other than donated organs. Therefore, research is currently being conducted on hepatic stem and progenitor cells. In general, progenitor cells are highly expandable in vitro, easily cryopreservable, and quite resistant to hypoxic conditions [5]. Hepatic progenitor cells (HPCs) mature rapidly into adult hepatocytes in quiescent liver [6] and have far greater regenerative capacity than do adult hepatocytes in retrorsine-treated liver [7]. However, very little functional analysis of transplanted HPCs has been performed, and it is currently unknown whether transplanted HPCs can improve liver dysfunction. In order to transplant fully functional cells, it may be necessary for immature cells to be matured in vitro prior to transplantation. Therefore, the development of an in vitro maturation system is important.

Many studies of the maturation of primitive hepatic endodermal cells in vitro have demonstrated the requirement for maturation of not only soluble factors, such as fibroblast growth factors [8], oncostatin M [9], and hepatocyte growth factor [10], but also cell-cell contact between parenchymal cells and nonparenchymal cells [11-14]. Recently, Nagai et al. reported that cell-cell contact between hepatic stellate cells (HSCs) and liver epithelial cells induced the differentiation of the liver epithelial cells into a hepatocytic lineage [11]. Nitou et al. reported that the coexistence of fetal mouse hepatoblasts and nonparenchymal cells was essential for their mutual survival, proliferation, differentiation and morphogenesis [12].

A system to enrich mouse fetal HPCs have been designed previously [15]. In this system, fetal HPCs are enriched by the formation of cell aggregates, which is dependent upon homophilic binding of cell adhesion molecules such as E-cadherin. This system enables us to enrich the viable HPCs while limiting cell damage. Examining the antigenic profiles of HPCs is crucial in order to isolate only the HPCs. Therefore, it is important to identify and characterize the cell populations contained in the cell aggregates and to examine the interactions among these populations.

Embryonic stem (ES) cells, pluripotent cells derived from the inner cell mass of blastocysts [33], have the ability to differentiate in vitro into a variety of cell lineages, including neurons [34], cardiomyocytes [35], and insulin-producing cells [36]. ES cell-derived hepatocytes are anticipated as potential sources of therapeutic cells for the treatment of liver diseases [37-39]. These cells may also be useful to facilitate drug discovery. To realize these goals, however, it is necessary to be able to produce mature hepatocytes entirely in vitro. Recent studies have demonstrated that ES cells can differentiate into hepatocyte-like endodermal cells in vitro, but it has been difficult to regulate the spontaneous differentiation of these pluripotent cells [40-43]. Under previously established culture conditions, the efficiency of differentiation into hepatocytes was not evaluated by the visualization of hepatic lineage cells using suitable markers. It was previously reported that albumin-producing hepatocyte-like cells could be differentiated from mouse ES cells expressing green fluorescent protein (GFP) under the control of the albumin promoter/enhancer [45]. ES cell-derived immature hepatocyte-like cells could be isolated using alpha-fetoprotein (AFP) as a marker [46]. Both albumin and AFP are produced by extraembryonic endodermal cells, such as cells of the primitive and visceral endoderm, which can differentiate from ES cells [47-48]. Albumin- or AFP-producing cells, which are considered to be hepatocytes, derived from ES cells likely constitute the majority of extraembryonic endodermal cells. Thus, while these results are promising, it has not been definitively reported that mature hepatocytes can be differentiated from ES cells in vitro. In addition, definitive factors or molecular pathways responsible for the terminal differentiation of hepatocytes from embryonic endodermal cells during development have remained unclear.

### SUMMARY OF THE INVENTION

Therefore, there is a need for provision of a method of producing mature hepatocytes from ES cells entirely in vitro for developing therapeutic cells for treatment of liver diseases, as well as for facilitating drug discovery.

The present inventors have conducted extensive research, and found that mature hepatocytes could be produced from ES cells entirely in vitro via isolation of an endodermal cell population that included hepatic progenitor cells, and subsequent maturation of these cells using Thy1-positive cells as a feeder layer.

Thus, the present invention provides a method for producing mature hepatocytes from ES cells in vitro by cell-cell contact of CD49f-positive cells with Thy1-positive cells.

The present invention also provides a method for isolating CD49f-positive and Thy 1-positive cells from fetal HPCs, which uses cell-enrichment characterized by formation of fetal HPC cell aggregates in combination with cell-sorting means such as FACS.

Specifically, the present invention provides the following:

[1] a method for preparing a mature hepatocyte from an embryonic stem cell in vitro, comprising:

(a) culturing the embryonic stem cell so as to differentiate into an endodermal cell;

(b) isolating a population of the endodermal cell from a population of the differentiated cell; and

(c) culturing the isolated endodermal cell in the presence of a Thy1-positive mesenchymal cell,

[2] the method according to [1], wherein said culturing the embryonic stem cell is performed under serum- and feeder layer-free culture conditions,

[3] the method according to [1], wherein said endodermal cell population comprise a hepatic progenitor cell,

[4] the method according to [1], wherein said Thy1-positive mesenchymal cell is used as a feeder cell layer,

[5] the method according to [1], wherein said Thy1-positive mesenchymal cell is gp38-positive,

[6] the method according to [1], wherein said embryonic stem cell is derived from a mouse,

[7] the method according to [1], wherein said embryonic stem cell is transfected with a neomycin resistance construct which contains a Hyg/EGFP fusion protein gene under the control of an AFP promoter,

[8] the method according to [7], wherein said endodermal cell is an AFP-GFP-positive cell,

[9] a mature hepatocyte, which is prepared by the method according to [1],

[10] a method for preparing a CD49f-positive cell and/or a Thy1-positive cell from a fetal hepatic progenitor cell, comprising:

(a) enriching the fetal hepatic progenitor cell through formation of cell aggregate;

(b) dissociating the cell aggregate into single cells;

(c) labeling the dissociated cell with a labeled antibody including an antibody specific to CD49f and Thy1; and

(d) separating the labeled cell by cell separation means to isolate a CD49f-positive cell and/or a Thy1-positive cell,

[11] the method according to [10], wherein the step (b) of dissociating the cell aggregate into single cells comprises:

(e) inoculating the cell aggregate on a type I collagen-coated culture plate to form a monolayer colony; and

(f) incubating the cells adhered to the culture plate with trypsin-EDTA solution,

[12] the method according to [10], further comprising:

(g) separating the Thy l-positive cells into a gp38-positive and a gp38-negative fractions,

[13] the method according to [10], wherein said fetal hepatic progenitor cell is obtained from a fetal liver,

[14] the method according to [10], wherein said labeled antibody is labeled with a fluorescence dye,

[15] the method according to [10], wherein said cell separation means is a fluorescence-activated cell sorter,

[16] a method for preparing a mature hepatocyte in vitro, comprising:

coculturing a CD49f-positive cell with a Thy1-positive cell,

wherein said CD49f-positive cell and said Thy1-positive cell are derived from a fetal hepatic progenitor cell,

[17] the method according to [16], wherein said Thy1-positive cell is gp38-positive,

[18] the method according to [16], wherein said CD49f-positive cell and said Thy1-positive cell are prepared by the method according to [10],

[19] a method for treating a liver disease, comprising:

administering the mature hepatocyte according to [9] to a recipient, and

[20] a pharmaceutical composition for treating a liver disease, comprising the mature hepatocyte according to [9] and a pharmaceutically acceptable carrier.

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE SEVERAL VIEWS OF THE DRAWINGS

FIG. 1 shows a flow diagram illustrating the isolation of fetal HPCs and FACS analysis/cell sorting.

FIG. 2 shows a flow-cytometric fractionation of the cell aggregates. (A) The cell aggregates are composed of three fractions: a Thy1⁻-CD45⁻ fraction (R1), a Thy1⁺CD45⁻ fraction (R2), and a Thy1⁻CD45⁺ fraction (R3). (B-C) Expression of CD49f in each fraction (R1-R3). (B) The Thy1⁻CD45⁻ fraction (R1) is CD49f⁺. (C) The Thy1⁺CD45⁻ fraction (R2) is CD49f^{±}. (D) The Thy1⁻CD45⁺ fraction (R3) is CD49f^{+(low and high)}. (E) The CD49f⁺Thy1⁻CD45⁻ fraction (R1) expresses c-Met and (F) The CD49f^{±}Thy1⁺CD45⁻ fraction (R2) expresses c-Met more strongly than do the other fractions. (G) No cells in the cell aggregates express c-Kit. A dotted line in (B) to (G) shows the negative control. (H) The Thy1-positive mesenchymal cell population was separated into two fractions: a gp38-positive fraction (purity 97%) and a gp38-negative fraction (purity 94%).

FIG. 3 shows immunocytochemical analysis of sorted Thy1-positive cells. The figures show phase-contrast (A, D) and fluorescent images (B, C, E and F). (A) After 24-hour culture, Thy1-positive cells appeared morphologically to be of two cell types, one spindle-shaped, and the other, surrounded by black dotted line, having a more highly granulated cytoplasm. (B) Most Thy1-positive cells stained positive for α-SMA, and (C) the second cell type stained for desmin. A colony made up of the second cell type is indicated by a white dotted circle. (D) At day 5, round cells with large nuclei appeared and proliferated. These cells did not stain for either (E) α-SMA or (F) desmin. Colonies of this cell type (white dotted line) were surrounded by α-SMA- or desmin-positive cells. (Original magnification: A-D, ×400; E and F, ×200.) Immunocytochemical analysis of gp38 in Thy1-positive mesenchymal cells. Phase contrast (G) and the corresponding fluorescent microscopic (H) images of isolated Thy1-positive cells derived from fetal liver nine days after isolation. The Thy1-positive population contained gp38-positive (red) and -negative cells. Phase contrast (1, K) and fluorescent microscopic (J, L) images of the isolated gp38-positive (I, J) and -negative (K, L) Thy1-positive cells. Original magnifications: G and H, ×100; I-L, ×200.

FIG. 4 shows RT-PCR analysis and histogram plots of flow-cytometric analysis of Thy1-positive cells. (A) RT-PCR shows that Thy1-positive cells express desmin, α-SMA and vimentin mRNA, but not markers of endothelial cells and Kupffer cells (lane 1). Lane 2, E13.5 fetal liver; lane 3, adult liver. (B) Thy1-positive cells are CD31⁻, CD34⁻, Flk1⁻, CD16⁻, CD29⁺, CD44^{±}, CD105⁺, CD106^{±}, CD71⁺, and Sca-1^{±}. A dotted line shows the negative control. The expression of mesenchymal markers by the two mesenchymal cell populations. Immunocytochemical analysis of gp38-positive (C, E) and -negative (D, F) cells for α-SMA (C, D) and desmin (E, F). (G) RT-PCR analysis of gp38-positive (left) and -negative (right) cells for α-SMA, desmin, vimentin, GFAP, PECAM, Flk-1, VE-cadherin, CD34, CD16, Integrin β4, CFTR, PDGFR-β, nestin, integrin α8, and HPRT. Original magnifications: C-F, ×200.

FIG. 5 shows co-culture and separate culture of CD49f-positive cells and Thy1-positive cells. The figures show phase-contrast images (A-F). (Original magnification: A-C, ×200; D-F, ×400.) In co-culture, (A) CD49f-positive cells (surrounded by closed arrows) appeared to show an increase in intracellular granularity at day 3. The inset shows that the colonies surrounded by closed arrows consist of AFP-positive cells, which are the CD49f-positive cells. (B) These colonies were piled-up at their peripheries at day 7. (C) At day 14, the piled-up area was widely expanded in the colonies of CD49f-positive cells. The inset shows high magnification of the boxed area. (D) At day 10, a number of cells in the CD49f-positive colonies were positive for PAS staining in co-culture. In contrast, CD49f-positive cells cultured alone (E) or separately with Thy1-positive cells (F) were negative for PAS staining even at day 10. Cocultures of CD49f-positive cells and mesenchymal cells. (G) CD49f-positive cells cocultured with gp38-positive cells for seven days. Arrows indicate the binuclear cells. (H) CD49f-positive cells cocultured with gp38-negative cells for seven days. (I, J) PAS staining of CD49f-positive cells cocultured with gp38-positive (I) and -negative (J) cells. (K) BrdU incorporation by CD49f-positive cells under different culture conditions. 1: coculture with gp38-positive cells, 2: CD49f-positive cells alone, and 3: coculture with gp38-negative cells. Values are expressed as means±SD (n=3). *P<0.05. Original magnifications: G-J, ×200.

FIG. 6 shows relative mRNA expression levels analyzed by real-time RT-PCR. Quantified mRNA levels of (A) AFP, (B) TAT, and (C) TO, which were normalized against that of glyceraldehyde-3-phosphate dehydrogenase (GAPDH) for each total RNA preparation, are expressed as means±SD from triplicate assays. Gray bar, CD49f-positive cells cultured alone; black bar, CD49f-positive cells cultured separately with Thy1-positive cells; white bar, co-cultured cells. Lane 1, day 2; lane 2, day 7; lane 3, day 14. RT-PCR and real-time RT-PCR analysis of cocultured cells. (D) mRNA expression by CD49f-positive cells cocultured with gp38-positive (left) or -negative (right) cells for seven days. (E and F) Relative mRNA-expression levels of TAT (E) and TO (F) after normalization to GAPDH levels, as determined by real-time RT-PCR. The graphs represent the mean values±SD from triplicate assays (n=3). *P<0.05. Black bars, cocultures of CD49f-positive cells with gp38-positive or -negative cells for two days; White bars, coculture of CD49f-positive cells with gp38-positive or -negative cells for seven days.

FIG. 7 shows transmission electron microscopic views of the CD49f-positive cells and the Thy1-positive cells just after their separation, and CD49f-positive and Thy1-positive cells after 30 days of co-culture. (A) The CD49f-positive cells just after separation had large nuclei with developed nucleoli (open arrow) and a number of fat droplets (closed arrow), but had few intra-cytoplasmic organelles such as mitochondria and peroxisomes. (B) The Thy1-positive cells just after separation appeared to comprise two cell types: cells with either clear (right) or dark (left) cytoplasm. Both cell types had large nuclei, a large amount of open rough endoplasmic reticulum (closed arrow), and many microfilaments in the cytoplasm. (C) The co-cultured cells had many mitochondria (short arrow), possessed peroxisomes (long arrow) and tight junctions with desmosomes (arrowhead), and formed biliary canaliculi with microvilli (open arrow). (Original magnification: A, ×2000; B and C, ×3000.) Scale bar, 5 μm.

FIG. 8 shows differentiation of mouse ES cells under serum- and feeder layer-free conditions. (A-D) The figures display phase-contrast (left panels) and fluorescent (right panels) images. (A) Undifferentiated ES cells cultured on mouse embryonic fibroblasts (B) at day 5, (C) day 7, and (D) day 10 after the initiation of differentiation. (E-H) Immunocytological analysis of differentiated ES cells at day 8. (E) Green fluorescence represents the expression of the transfected GFP gene. (F) Red fluorescence indicates AFP expression. (G and H) The merged images of (E) and (F). Original magnifications: A-D, and H, ×400; E-G, ×200. (I) RT-PCR analysis of RNA samples extracted from undifferentiated ES cells and differentiated ES cells at days 2, 5, 7, and 10 after the initiation of differentiation. GFP, green fluorescent protein; AFP, alpha-fetoprotein, GAPDH, glyceraldehyde-3-phosphate dehydrogenase, and RT (−) as a negative control.

FIG. 9 shows flow cytometric analysis of differentiated ES cells. (A) Histogram plots of differentiated ES cells using the group 3 protocol. Histogram plots exhibit two peaks at day 5-6, and one peak of GFP fluorescence at day 7-8. A dotted line represents the undifferentiated ES cells. (B) The proportions of GFP-positive cells to the total cells are expressed as the means±standard deviation from triplicate assays.

FIG. 10 shows immunocytological analysis of GFP-positive (A-E) and GFP-negative (F-H) cells after sorting. (A) A phase-contrast image of the GFP-positive cell fraction seven days after sorting. (B) Green fluorescence represents transfected GFP gene expression, while (C) red fluorescence indicates AFP expression. Fourteen days after sorting, fluorescent immunocytological images were obtained of (D) albumin (green) and DAPI (blue) and (E) Foxa2 (red) staining. (F) A phase-contrast image of the GFP-negative cell fraction. (G) Green florescence represents GFP expression, while (H) red fluorescence indicates AFP expression. Original magnifications: ×200.

FIG. 11 shows coculture of GFP-positive cells with Thy1-positive cells. (A) A phase-contrast image of the isolated GFP-positive cells cocultured with Thy1-positive cells for seven days. (B) Piled-up colonies were positive for PAS staining following coculture. In contrast, either (C) GFP-positive cells or (D) Thy1-positive cells cultured alone were negative for PAS staining. Original magnifications: ×200.

FIG. 12 shows RT-PCR analysis. mRNA was extracted from undifferentiated ES cells (ES), Thy1-positive cells treated with mitomycin C (lane 1), GFP-positive cells cultured alone for one month (lane 2), GFP-positive cells cultured on a feeder layer of Thy1-positive cells for seven days (lane 3), E13.5 fetal livers (FL), and adult livers (AL). TAT, tyrosine amino transferase; TO, tryptophan 2,3-dioxygenase; G6P, glucose-6-phosphatase, GAPDH, glyceraldehyde-3-phosphate dehydrogenase, and RT (−) as a negative control.

FIG. 13 shows transmission electron microscopic views of cocultured cells (A-C). (A) The cells of the piled-up colonies possessed tight junctions with desmosomes (closed arrow). These cells occasionally formed biliary canaliculi (open arrow). These cells exhibited well-developed mitochondria (Mt) and rough endoplasmic reticulum (rER) and large numbers of peroxisomes (Pr) and (B) glycogen granules (GI). (C) A portion of the cells were binucleate. (D) The isolated GFP-positive cells cultured alone for 30 days. Original magnifications: A, D, ×3000; B, C, ×2000.

FIG. 14 shows ammonia clearance activity of the cultured cells. AFP-GFP-positive cells cultured alone exhibited low activity to remove ammonia from the culture media. Cocultured cells displayed an approximately two-fold greater metabolizing activity than that of AFP-GFP-positive cells cultured alone. Thy1-positive cells cultured alone did not possess any activity to metabolize ammonia.

### DETAILED DESCRIPTION OF THE INVENTION

The following detailed description of the invention is provided to aid those skilled in the art in practicing the present invention. This detailed description should not be construed to limit the present invention, as modifications of the embodiments disclosed herein may be made by those of ordinary skill in the art without departing from the spirit and scope of the present invention. Throughout this disclosure, various publications, patents, and published patent specifications are referenced by citation. The disclosure of these publications, patents, and published patents are hereby incorporated by reference in their entirety into the present disclosure.

The practice of the present invention will employ, unless otherwise indicated, conventional techniques of immunology, molecular biology, microbiology, cell biology and recombinant DNA, which are within the skill of the art. See, e.g., Sambrook, et al. MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G R. Taylor eds. (1995)), Harlow and Lane, eds. (1988) ANTIBODIES, A LABORATORY MANUAL, and ANIMAL CELL CULTURE (R. I. Freshney, ed. (1987)).

Definitions

As used in the specification and claims, the singular form “a”, “an”, and “the” include plural references unless the context clearly dictates otherwise. For example, the term “a cell” includes a plurality of cells, including mixtures thereof A cell is of “ectodermal”, “endodermal” or “mesodermal” origin, if the cell is derived, respectively, from one of the three germ layers, i.e., ectoderm, the endoderm, or the mesoderm of an embryo. The ectoderm is the outer layer that produces the cells of the epidermis, and the nervous system. The endoderm is the inner layer that produces the lining of the digestive tube and its associated organs. The middle layer, mesoderm, gives rise to several organs, including but not limited to heart, mesothelium, and urogenital system, connective tissues (e.g., bone, muscles, tendons), and the blood cells.

The terms “mammals” or “mammalian” refer to warm blooded vertebrates which include but are not limited to humans, mice, rats, rabbits, simians, sport animals, and pets.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins, and modifications of the immunoglobulin molecule that comprise an antigen recognition site of the required specificity.

The term “antigen” is a molecule which can include one or more epitopes to which an antibody can bind. An antigen is a substance which can have immunogenic properties, i.e., induce an immune response. Antigens are considered to be a type of immunogen. As used herein, the term “antigen” is intended to mean full length proteins as well as peptide fragments thereof containing or comprising one or a plurality of epitopes.

The terms “surface antigen(s)” and “cell surface antigen” are used interchangeably herein and refer to the plasma membrane components of a cell. These components include, but are not limited to, integral and peripheral membrane proteins, glycoproteins, polysaccharides, lipids, and glycosylphosphatidylinositol (GPI)-linked proteins. An “integral membrane protein” is a transmembrane protein that extends across the lipid bilayer of the plasma membrane of a cell. A typical integral membrane protein consists of at least one membrane spanning segment that generally comprises hydrophobic amino acid residues. Peripheral membrane proteins do not extend into the hydrophobic interior of the lipid bilayer and they are bound to the membrane surface by noncovalent interaction with other membrane proteins. GPI-linked proteins are proteins which are held on the cell surface by a lipid tail which is inserted into the lipid bilayer.

The term “monoclonal antibody” as used herein refers to an antibody composition having a substantially homogeneous antibody population. It is not intended to be limited as regards to the source of the antibody or the manner in which it is made (e.g. by hybridoma or recombinant synthesis). Monoclonal antibodies are highly specific, being directed against a single antigenic site. In contrast to conventional (polyclonal) antibody preparations which typically include different antibodies directed against different determinants (epitopes), each monoclonal antibody is directed against a single determinant on the antigen.

1. Isolation of CD49f-Positive and Thy1-Positive Cells

We have previously reported a method for enriching mouse fetal HPCs that relies on formation of cell aggregates [15]. Here, we used this system in combination with cell separation procedure such as FACS to isolate CD49f-positive cells and Thy1-positive cells.

That is, using the cell aggregate method in combination with cell separation procedure such as FACS, we identified two cell populations, one CD49f-positive and the other Thy1-positive, in our enriched mouse fetal HPC cell aggregates. The CD49f-positive cells were primitive hepatic endodermal cells. The Thy1-positive cells are probably of mesenchymal origin and promoted the maturation of CD49f-positive cells by cell-cell contact. Thus, a large number of CD49f-positive primitive hepatic endodermal cells can be isolated using our cell aggregate method and FACS sorting.

Therefore, in one aspect of the present invention, there is provided a method for preparing a CD49f-positive cell and/or a Thy1-positive cell from a fetal hepatic progenitor cell, comprising:

(a) enriching the fetal hepatic progenitor cell through formation of cell aggregates;

(b) dissociating the cell aggregates into single cells;

(c) labeling the dissociated cell with a labeled antibody including an antibody specific to CD49f and Thy1; and

(d) separating the labeled cells by cell separation means to isolate a CD49f-positive cell and/or a Thy1-positive cell.

In a preferred embodiment, the fetal hepatic progenitor cells are derived from mammalian feral liver (e.g., E13.5 fetal liver).

In a preferred embodiment, the step (b) of dissociating the cell aggregates into single cells comprises: (e) inoculating the cell aggregates on a type I collagen-coated culture plate to form monolayer colonies; and (f) incubating the cells adhered to the culture plate with trypsin-EDTA solution such that the adhered cells are dissociated. With this procedure, dissociation of the cell aggregates into single cells is facilitated.

In a preferred embodiment, the method further comprises: (g) separating the Thy1-positive cell into a gp38-positive and a gp38-negative fractions.

In order to isolate the dissociated cells, cell separation means such as, but not limited to, flow cytometory (e.g., fluorescence-activated cell sorter) can be used. Prior to being subjected to the cell separation means, each dissociated cell is typically labeled with a labeled antibody. In a preferred embodiment, the labeled antibody is labeled with a fluorescence dye. Typical antibodies used for the present invention are exemplified by, but not limited to, the following antibodies: (all diluted at 1:100) anti-Thy1-fluorescein isothiocyanate (FITC) (Immunotech, Marseille, France), CD49f (integrin α6)-phycoerythrin (PE), CD45 (leukocyte common antigen)-allophycocyanin (APC), CD29 (integrin β1)-FITC, CD16-FITC, CD31 (PECAM-1)-FITC, CD34-FITC, Flk1 (VEGF-R2)-PE, CD44-APC, CD106-FITC, c-Kit-APC, CD71 (transferrin receptor)-FITC, and Sca-1-PE monoclonal antibody (mAb) (Pharmingen, San Jose, Calif., USA). For anti-CD105 (endoglin) (Pharmingen, San Jose, Calif., USA) and anti-c-Met (hepatocyte growth factor receptor) mAb (Santa Cruz Biotechnology, Inc., Santa Cruz, Calif.) diluted at 1:100, the second antibody was biotin-conjugated anti-mouse immunoglobulin G (IgG) diluted at 1:100, and visualization was performed using streptavidin-APC (Pharmingen, San Jose, Calif., USA) diluted at 1:100.

In yet another aspect of the present invention, there is provided a method for preparing a mature hepatocyte in vitro, comprising: coculturing a CD49f-positive cell with a Thy1-positive cell, wherein said CD49f-positive cell and said Thy1-positive cell are derived from a fetal hepatic progenitor cell.

In a preferred embodiment, the CD49f-positive cell and the Thy1-positive cell for use in this aspect of the present invention are prepared by the method described above.

2. Production of Mature Hepatocytes from ES Cells In Vitro

We have developed a method for isolating ES cell-derived AFP-producing cells, which cells could maturate into hepatocyte-like cells in vitro by coculture with Thy1-positive fetal liver cells.

Therefore, in another aspect of the present invention, there is provided a method for preparing a mature hepatocyte from an embryonic stem cell in vitro, which comprises:

(a) culturing the embryonic stem cell so as to differentiate into an endodermal cell;

(b) isolating a population of the endodermal cell from a population of the differentiated cell; and

(c) culturing the isolated endodermal cell in the presence of a Thy1-positive mesenchymal cell.

The embryonic stem cell can be derived from mammals such as humans, mice, rats, rabbits, simians, pigs, horses, sport animals, pets or the like. As one of preferable examples, mouse ES cells derived from C57BL/6 mice can be used for the purpose of the present invention.

The embryonic stem cell can be cultured under appropriate culture conditions. A non-limiting example of culture conditions is, for example, Dulbecco's modified essential medium (DMEM) (Sigma) supplemented with 20% fetal bovine serum (FBS) (HyClone, Logan, Utah), 0.1 mM 2-mercaptoethanol (Sigma), nonessential amino acids (Sigma), 1 mM sodium pyruvate (Sigma), and 1000 U/ml leukemia inhibitory factor (LIF) (ESGRO, Chemicon International Inc., Temecula, Calif.) on a mouse embryonic fibroblast feeder layer treated with mitomycin C (Wako Pure Chemical Co., Osaka, Japan), as indicated in the EXAMPLE below.

To improve the efficiency of endodermal differentiation, it is preferred that the ES cells are transferred into serum- and feeder layer-free culture conditions prior to differentiation. In comparison to previously described methods of forming EBs, this method spread the differentiated ES cells over culture dishes in a monolayer, which makes subsequent procedure such as immunocytological and flow cytometric analysis more simple and effective.

In a preferred embodiment, a Thy1-positive, gp38-positive mesenchymal cell is used for culturing with the isolated endodermal cell.

In a preferred embodiment, to facilitate selection of cells of interest, the embryonic stem cell is transfected with a neomycin resistance construct, which contains a Hyg/EGFP fusion protein gene under the control of an AFP promoter. Isolation of the endodermal cells from the differenciated cells can then be performed using cell separation technique such as, but not limited to, flow cytometry or the like. The endodermal cells of interest can then be selected as AFP-GFP-positive cells. In a typical embodiment, most of the endodermal cell population corresponds to hepatic progenitor cells.

The isolated endodermal cells are then subjected to cell-to-cell contact with Th1-positive mesenchymal cells so as to maturate to hepatocytes. Such cell-to-cell contact can be performed by, for example, coculturing the isolated endodermal cells with Thy-positive inesenchymal cells. In a preferred embodiment, Thy1-positive mesenchymal cells are used as a feeder cell layer. Thy1-positive mesenchymal cells can be prepared from mammalian fetal liver (e.g., mouse fetal liver). Although Thy1-positive mesencymal cells can be prepared by any appropriate method, Thy1-positive mesencymal cells can be typically prepared by the method described in EXAMPLE 1, which combines use of below-listed antibodies with flow cytometory: anti-Th1-fluorescein isothiocyanate (FITC) (Immunotech, Marseille, France), anti-CD49f-phycoerythrin (PE), and anti-CD45-PE (BD Biosciences Pharmingen, San Diego, Calif.). The fluorescent dyes for use with the antibodies can be appropriately modified by those skilled in the art.

3. Utility of the Present Invention

In still another aspect of the present invention, there is provided a mature hepatocyte, which is prepared by the method described in section 2 above.

The mature hepatocytes produced by the method of the present invention are useful as ES cell-derived donor cells for the therapy of liver diseases, which requires the generation of essentially pure endodermal cells and the subsequent maturation of these cells into functional hepatocytes in vitro.

The present invention also provides a pharmaceutical composition for use in implant therapy. The composition includes the mature hepatocytes of this invention in a pharmaceutically acceptable carrier, auxiliary or excipient. The composition may also contain one or more types of cells differentiated from fetal progenitor cells derived from mammalian fetal liver.

The present invention also provides a therapeutic composition or a kit for the treatment of a disease, disorder or abnormal physical state such as the above. The composition or kit includes one or more types of cells including the mature hepatocytes of the present invention, or other type of cells (e.g., immature hepatic cells) differentiated from mammalian fetal liver.

A method of treating an individual suffering from a liver disease is also included within this invention. The method includes implanting the mature hepatocytes produced by the method of the present invention, into the liver, or other damaged tissues of the individual. In this method, the mammal may be a human who is suffering from a liver disease, disorder (such as liver cirrhosis) or abnormal physical state. In another case, the mammal is a human and is not suffering from a liver disease. In such a case, the method includes implanting the mature hepatic cells produced by the method of the present invention, into a second human who is suffering from a liver disease. The liver disease may be one selected from a group consisting of fluminant/acute/chronic heptatitis, autoimmune hepatitis, liver cirrhosis, congenital defect of enzymes, and liver cancer.

The present invention also provides a kit for preparing mature hepatocytes, which kit comprises CD49f-positive cells and Thy1-positive cells derived from fetal hepatic progenitor cells. The kit may also contain culturing media suitable for culturing the CD49f-positive and the Thy1-positive cells. The kit may also contain an indication describing the procedure on how to prepare mature hepatocytes by using the kit.

(i) Uses of Mature Hepatocytes for Cell Therapy

In one use, mature hepatic cell lines are used for cell therapy. Transplantation of mature hepatocytes is one such example of cell therapy. In cases where different types of hepatocytes are desired, transplantation of mature hepatocytes may be employed because the hepatocytes of this invention are multipotent and can differentiate into cholangiocytes. To practice this use, mature hepatocytes are isolated and cultured in basal nutrient, nutrient-defined media using the methods disclosed. Mature hepatocytes are grown on type-I collagen-coated tissue culture plates to obtain mature hepatic cell clusters. Mature hepatic cell clusters are grown under standard incubation conditions for about half a day to at least about 1 cell cycle passage, more preferably for at least about 2 cell cycle passage, most preferably at least about 3 cell cycle passages. Mature hepatic cell aggregates can then be administered to a recipient and allowed to differentiate. In an alternative, mature hepatic cell aggregates can be used as cellular carriers of gene therapy wherein mature hepatocytes are transfected with one or more genes and enclosed in a delivery device and then administered to a recipient. In another embodiment, mature hepatic cell aggregates are used in a device which contains cells and limits access from other cells to limit immune system responses. The recipient can be human or other mammalians.

(ii) Uses of Mature Hepatocytes to Make Human Tissue Models

Another use for mature hepatocytes is to generate human liver tissue models in non-human mammals. A human liver tissue models can be employed to study multiple facets of liver development or liver carcinogenesis, an important area of hepatic cancer research. Mature hepatic cell spheres are placed on top of mesenchymal tissue to form grafting recombinants. To form grafting recombinants, about 1 to 15 mature hepatic cell spheres, more preferably about 5 to 8 spheres, are placed on top of mesenchymal tissue. The mesenchymal tissue may be either hepatic or non-hepatic tissue and may be derived from a different species from which mature hepatocytes are isolated. In an example, human mature hepatocytes are placed on top of rat mesenchymal urogenital tissue to form a graft recombinant. A skilled artisan may determine the optimal combination for human mature hepatic cell growth in a stepwise fashion, by first isolating human mature hepatocytes using the methods disclosed herein and then combining with mesenchymal tissue from different organs. In some embodiments, a different species, e.g. rat, is used as a source for mesenchymal tissue in combination with human mature hepatocytes. The use of heterologous species allows human-specific markers to be used to determine the identity of differentiated human hepatocytes. The likelihood of false positives is reduced if rat mesenchymal tissue is used. In a preferred embodiment, about 1 to 12 mature hepatic cell spheres, even more preferably about 5 to 8 mature hepatic cell spheres, are placed on top of rat urogenital mesenchymal cells. Preferably, about 1×10⁴ to about 5×10⁶ mesenchymal cells are used. Even more preferably, about 2×10⁵ to about 5×10⁵ mesenchymal cells are used. A graft recombinant comprising mature hepatic cell spheres placed on mesenchymal tissue is then placed under the kidney capsule of a recipient mammal. Possible recipient mammals include but are not limited to mice and rats. Typically in graft situations, donor tissue is vulnerable to attack by the recipient's immune system. To alleviate graft rejection, several techniques may be used. One method is to irradiate the recipient with a sub-lethal dose of radiation to destroy immune cells that may attack the graft. Another method is to give the recipient cyclosporin or other T cell immunosuppressive drugs. With the use of mice as recipient mammals, a wider variety of methods are possible for alleviating graft rejection. One such method is the use of an immunodeficient mouse (nude or severe combined immunodeficiency or SCID). In one embodiment, human mature hepatic cell spheres are placed on rat urogenital mesenchymal tissue and placed under the kidney capsule of an immunodeficient mouse. The graft recombinant remains in the recipient for about 1 to about 52 weeks, preferably about 5 to about 40 weeks, and even more preferably about 6 to about 8 weeks before the grafts are harvested and analyzed for mature hepatic cell differentiation. In some cases, a small portion of the graft is needed for analysis. Markers specific for the hepatic surface epithelial cell include, but are not limited to, albumin may be utilized to confirm the identity of the differentiated mature hepatocytes. Non-limiting methods of confirming markers are immunohistochemical analysis, RT-PCR, and flow cytometry. Another method of identifying the differentiated mature hepatocytes and assessing the success of the transplantation is to stain for the presence of glucose in hepatic surface epithelial cells. These markers can be used separately or in combination with each other. In addition, a combination of one or more of these markers may be used in combination with cell morphology to determine the efficacy of the transplantation.

In one embodiment, human hepatic model can be generated in a SCID (severe combined immunodeficiency) mouse. The human hepatic model can be made by utilizing the human mature hepatocytes isolated and cultured with methods disclosed herein and using the human mature hepatocytes to make graft recombinants. Graft recombinants are then placed under the kidney capsule of mice. After about 1 to 10 weeks, preferably about 6 to 8 weeks after implantation under the kidney capsule, the graft or portion thereof is harvested and analyzed by immunohistochemistry. Markers specific to hepatic surface epithelial cells include, but are not limited to, albumin. Markers specific to hepatic surface epithelial cells are used to analyze the efficacy of the tissue model system. Alternatively, markers specific for differentiated mature hepatocytes are used. Non-limiting examples of these markers are: TAT, TO, and G6P. Yet another way to assess the results of mature hepatic cell differentiation is by morphology. Hepatic surface epithelial cells have the appearance of flat or columnar epithelial cells.

(iii) Uses of Mature Hepatocytes in Bioassays

The mature hepatocytes disclosed herein can be used in various bioassays. In one use, the mature hepatocytes are used to determine which biological factors are required for differentiation. By using the mature hepatocytes in a stepwise fashion in combination with different biological compounds (such as hormones, specific growth factors, etc.), one or more specific biological compounds can be found to induce differentiation of hepatic progenitor cells to mature hepatocytes. Employing the same stepwise combinations, one or more specific biological compound can be found to induce differentiation of hepatic progenitor cells to cholangiocytes. Other uses in a bioassay for mature hepatocytes are differential display (i.e. mRNA differential display) and protein-protein interactions using secreted proteins from mature hepatocytes. Protein-protein interactions can be determined with techniques such as yeast two-hybrid system. Proteins from mature hepatocytes can be used to identify other unknown proteins or other cell types that interact with mature hepatocytes. These unknown proteins may be one or more of the following: growth factors, hormones, enzymes, transcription factors, translational factors, and tumor suppressors. Bioassays involving mature hepatocytes and the protein-protein interaction these cells form and the effects of protein-protein or even cell-cell contact may be used to determine how surrounding tissue, such as mesenchymal tissue, contributes to mature hepatic cell differentiation.

### EXAMPLES

Hereinafter the present invention will be described in more detail with reference to EXAMPLES but the scope of the present invention should not be deemed to be limited thereto.

### Example 1

Materials and Methods

Animals

C57BL/6J Jms Slc mice were obtained from SLC (Hamamatsu, Japan). Animals were maintained at a constant temperature of 18° C. to 20° C. and in a 12-hour-light/12-hour-dark cycle. They were housed at, and all animal experimental procedures were performed according to, the Animal Protection Guidelines of Kyoto University.

Isolation and Culture of Fetal HPCs

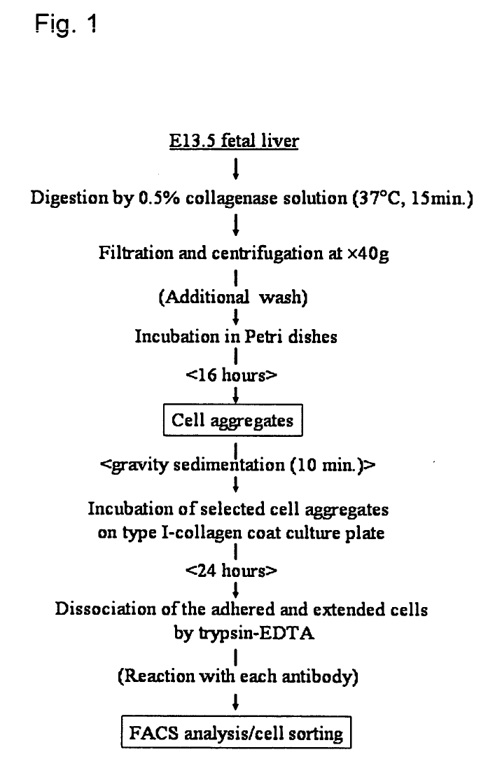

Fetal HPCs were obtained from E13.5 fetal livers, and were enriched by formation of cell aggregates. The isolation and culture of the cell aggregates was performed as described previously [15]. Dissociating the cell aggregates into single cells is technically difficult. Therefore, cell aggregates selected by gravity sedimentation were inoculated on type-I collagen-coated culture plates (Becton Dickinson Co., Ltd., Lincoln Park, N.J.). After 24 hours of incubation, the aggregates adhered to the plates and extended as monolayer colonies. After removing hematopoietic cells by washing twice with phosphate buffered saline (PBS), adherent cells were incubated with trypsin-EDTA solution (Sigma Chemical Co., Ltd., St. Louis, Mo., USA) for 12 minutes. The dissociated cells were washed three times with PBS containing 3% fetal calf serum (FCS) (ICN, Aurora, Ohio, USA) and were used for fluorescence-activated cell sorter (FACS) analysis or FACS sorting. A flow diagram describing the formation of cell aggregates and FACS analysis/cell sorting is shown in FIG. 1.

FACS Analysis

The dissociated cells were incubated with each antibody at 4° C. for 30 minutes, washed three times and resuspended in 3% FCS-PBS. The following antibodies were used, all diluted at 1:100: anti-Thy1-fluorescein isothiocyanate (FITC) (Immunotech, Marseille, France), CD49f (integrin Δ6)-phycoerythrin (PE), CD45 (leukocyte common antigen)-allophycocyanin (APC), CD29 (integrin β1)-FITC, CD16-FITC, CD31 (PECAM-1)-FITC, CD34-FITC, Flk1 (VEGF-R2)-PE, CD44-APC, CD106-FITC, c-Kit-APC, CD71 (transferrin receptor)-FITC, and Sca-1-PE monoclonal antibody (mAb) (Pharmingen, San Jose, Calif., USA). For anti-CD105 (endoglin) (Pharmingen, San Jose, Calif., USA) and anti-c-Met (hepatocyte growth factor receptor) mAb (Santa Cruz Biotechnology, Inc., Santa Cruz, Calif.) diluted at 1:100, the second antibody was biotin-conjugated anti-mouse immunoglobulin G (IgG) diluted at 1:100, and visualization was performed using streptavidin-APC (Pharmingen, San Jose, Calif., USA) diluted at 1:100. Then, the cells were analyzed with a FACSCalibur (Beckton Dickinson, San Jose, Calif., USA). Gating was implemented based on isotypic control staining profiles.

Cell Sorting by FACS and Culture of the Separated Cells

The dissociated cells were incubated with Thy1-FITC, CD49f-PE, and CD45-APC mAb at 4° C. for 30 minutes and prepared as described above. Then, the cells were separated using a FACSVantage (Becton Dickinson, San Jose, Calif., USA). After separation, the collected cells were washed twice with 3% FCS-PBS, resuspended in Dulbecco's modified essential medium (Gibco BRL, Grand Island, N.Y.) with 10% FCS, 20 mmol/L HEPES, 25 mmol/L NaHCO₃, 0.5 mg/L insulin, 10⁻⁷ mol/L dexamethasone (Wako Pure Chemical, Osaka, Japan), 10 mmol/L nicotinamide (Wako Pure Chemical, Osaka, Japan), 2 mmol/L L-ascorbic acid phosphate (Wako Pure Chemical, Osaka, Japan), 20 μg/L deleted form of hepatocyte growth factor (kindly provided by Snow Brand Product Co., Osaka, Japan), 100 units/mL penicillin G, and 0.2 mg/mL streptomycin. Then, the cells were inoculated on type-I collagen-coated 24-well plates (Becton Dickinson Co., Ltd., Lincoln Park, N.J.) at a density of 2×10⁴/well. To evaluate the interaction between the two separated cell fractions, co-culture was performed as follows. In method 1, a mixture of both cell fractions (1:1 in cell density) was inoculated on type-I collagen-coated 24-well plates. In method 2, both cell fractions were cultured separately on type-I collagen-coated 24-well plates using BIOCOAT Cell Culture Inserts (Becton Dickinson Co., Ltd., Lincoln Park, N.J.). After 16 hours, the culture media were changed, and thereafter, the media were changed every 2-3 days.

Immunocytochemistry

Immunocytochemistry for α-fetoprotein (AFP), albumin (ALB), and cytokeratin19 (CK19) was performed as previously described [15]. For immunocytochemistry of desmin and alpha-smooth muscle actin (α-SMA), the cultured cells were washed twice with PBS and fixed in 3.3% formalin for 12 minutes at room temperature. Nonspecific binding was blocked with 10% skim milk (Snow Brand Product Co., Gunma, Japan) and 0.4% bovine serum albumin (Sigma-Aldrich Chemie Co., Ltd., Steinheim, Germany) in 0.1% saponin (Sigma Chemical Co., Ltd., St. Louis, Mo., USA) in PBS. Then, endogenous avidin and biotin were blocked with an avidin/biotin blocking kit (Vector Laboratories, Inc., Burlingame, Calif.). Subsequently, the cells were incubated with the primary antibodies for 16 hours at 4° C. followed by incubation with the biotin-conjugated secondary antibody for 30 minutes at 37° C. The primary antibodies were anti-human A-SMA (DAKO Japan, Kyoto, Japan) diluted at 1:200 and anti-human desmin (DAKO Japan, Kyoto, Japan) diluted at 1:100. The secondary antibody was biotin-conjugated anti-mouse IgG (DAKO Japan, Kyoto, Japan) diluted at 1:500, and visualization was performed using streptavidin-conjugated Texas Red-X (Molecular Probes, Inc., Eugene, Oreg.). Nuclear counterstaining was performed with 4′,6-diamidino-2-phenylindole. The signal was detected using a fluorescence microscope (Axiovert 135, Carl Zeiss Vision Co., Ltd., Hallbergmoos, Germany).

Reverse-Transcription Polymerase Chain Reaction (RT-PCR)

Total RNA was extracted from the separated cells just after sorting, E13.5 fetal liver and adult liver using an RNeasy kit (QIAGEN, Chatsworth, Calif., USA) according to the manufacturer's instructions. Complementary DNA was synthesized from total RNA using the Omniscript RT kit (QIAGEN, Chatsworth, Calif., USA) and amplified using specific primer pairs and AmpliTaq Gold DNA polymerase (Perkin Elmer, Foster City, Calif., USA). The PCR conditions were as follows: 95° C. for 15 minutes, followed by 30 cycles of 94° C. for 15 seconds, 60° C. for 30 seconds, and 72° C. for 1 minute, and a final extension at 72° C. for 10 minutes. Desmin, α-SMA, and vimentin were used as mesenchymal cell markers. VE-cadherin, PECAM, and Flk1 were used as endothelial cell markers. CD16 was used as a Kupffer cell marker. Primers used for amplification are listed in Table 1.

TABLE 1
Primer Sequences Used

| | |
|---|---|
| Desmin | 5′-GCTATCAGGACAACATTGCG-3′ |
| | 5′-GTTGTTGCTGTGTAGCCTCG-3′ |
| α-SMA | 5′-CTATTCAGGCTGTGCTGTTCC-3′ |
| | 5′-GGACCTCTTCTCGATGCTGA-3′ |
| Vimentin | 5′-TAGCAGGACACTATTGGCCG-3′ |
| | 5′-CTGTTGCACCAAGTGTGTGC-3′ |
| VE-cadherin | 5′-AGGCTGAATACAAGATCGTGG-3′ |
| | 5′-GGTCTGTCTCAATGGTGAAGG-3′ |
| PECAM | 5′-CCACTTCTGAACTCCAACAGC-3′ |
| | 5′-CCAACATGAACAAGGCAGC-3′ |
| Flk-1 | 5′-TCTTTCGGTGTGTTGCTCTG-3′ |
| | 5′-TAGGCAGGGAGAGTCCAGAA-3′ |
| CD16 | 5′-CCACAACTGGAGTTCCATCC-3′ |
| | 5′-TTGTTCCTCCAGCTATGGCACC-3′ |
| AFP | 5′-ACAGGAGGCTATGCATCACC-3′ |
| | 5′-TGGACATCTTCACCATGTGG-3′ |
| TO | 5′-GCTCAAGGTGATAGCTCGGA-3′ |
| | 5′-GGAACTCTGCCATCTGTTCC-3′ |
| TAT | 5′-TCCAGGAGTTCTGTGAACAGC-3′ |
| | 5′-AGTATATGGTGCCTGCCTGC-3′ |
| β-actin | 5′-TGGAGAAGAGCTATGAGCTGC-3′ |
| | 5′-GATCCACATCTGCTGGAAGG-3′ |

Real-Time RT-PCR

mRNA expression was quantified by real-time RT-PCR using ABI Prism 7700 (Applied Biosystems, FosterCity, Calif., USA). One-step RT-PCR reactions were performed in 96-well plates containing, in each well, 100 ng total RNA together with 0.1 μmol/L each of the sense and antisense primers and 0.2 μmol/L probe in a total volume of 25 μl. All reactions were run in triplicate. After the RT reaction at 48° C. for 30 minutes, the reaction mixtures were heated at 95° C. for 10 minutes, followed by 40 cycles at 95° C. for 15 seconds and 60° C. for 1 minute. The comparative threshold cycle (CT) method against the expression level of glyceraldehydes-3-phosphate dehydrogenase was used to determine relative quantities. Tyrosine amino transferase (TAT) was used as a perinatal hepatocyte marker gene, and tryptophan oxygenase (TO) was used as a mature hepatocyte marker gene. mRNA from fetal and adult liver was used as a positive control for AFP, TAT, and TO expression. Primers and probes used are listed Table 2.

TABLE 2
TaqMan Probe and Primer Sequences

| | |
|---|---|
| AFP | 5′-ATCGACCTCACCGGGAAGAT-3′ |
| | 5′-GAGTTCACAGGGCTTGCTTCA-3′ |
| | 5′-FAM-AATGTCGGCCATTCCCTCACCACAG-TAMRA-3′ |
| TAT | 5′-TGACGAGTGGCTGCAGTCA-3′ |
| | 5′-TGACCTCAATCCCCATAGACTCA-3′ |
| | 5′-FAM-TGGACAGAAGATCCTCATTCCGAGGC-TAMRA-3′ |
| TO | 5′-GGTGAACGACGACTGTCATACC-3′ |
| | 5′-CATGAGCGTGTCAATGTCCATA-3′ |
| | 5′-FAM-TACAGGGAAGAGCCTCGATTCCAGGTC-TAMRA-3′ |

Periodic Acid-Schiff (PAS)-Staining Analysis of Cultured Cells

To examine whether the cultured cells produced and stored glycogen, as an indicator of functional maturation, PAS staining was performed. The cultured cells were fixed in 3.3% formalin for 12 minutes, and intracellular glycogen was stained using a PAS staining solution (Muto Pure Chemicals Co., Ltd., Tokyo, Japan) according to the standard protocol.

Transmission Electron Microscopy

CD49f-positive cells and Thy1-positive cells were taken either immediately after their separation or after they had been co-cultured for 30 days, and they were fixed in 2% glutaraldehyde-PBS for 1 hour, postfixed in 2% osmium tetroxide, and embedded in Epon-812 resin. Ultra-thin sections were cut using an ultra-microtome and were stained with uranyl acetate. The sections were examined by transmission electron microscopy (H-7000, Hitachi, Tokyo, Japan).

Results

Flow-Cytometric Fractionation of Cell Aggregates

We found that approximately 50% of the cell aggregates were composed of CD49f-positive cells, which expressed c-Met but not c-Kit.

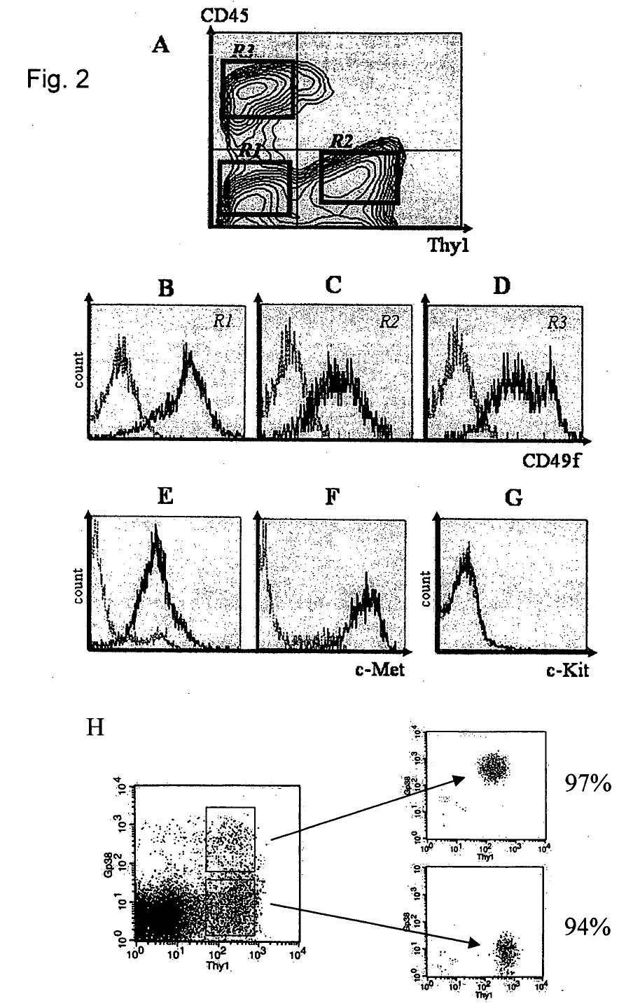

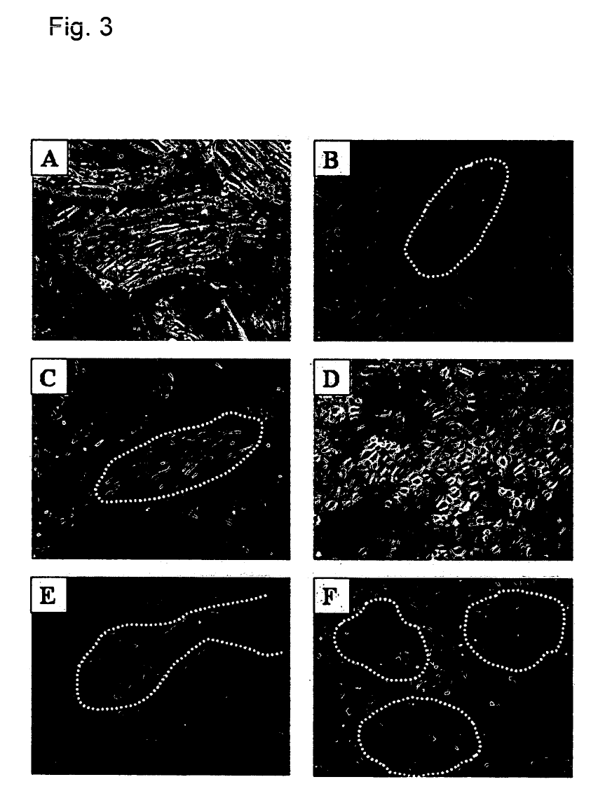

Specifically, FACS analysis using antibodies against CD49f, Thy1, and CD45 determined that three major populations existed in the cell aggregates: (1) CD49f⁺Thy1⁻CD45⁻ cells (CD49f-positive cells) (48.51±7.34%); (2) CD49f^{±}Thy1⁺CD45⁻ cells (Thy1-positive cells) (23.79±7.92%); and (3) CD49f^{+(low and high)}Thy1⁻CD45⁺ cells (CD45-positive cells) (24.78±8.18%) (FIGS. 2A-2D). Both CD49f- and Thy1-positive cells expressed c-Met, the latter more strongly (FIGS. 2E and 2F). However, none of the cells in the aggregates expressed c-Kit (FIG. 2G). Because CD45 is a common leukocyte antigen, and the CD45-positive cells isolated by FACS sorting did not attach to the culture dish, but remained floating, we believe that the CD45-positive cells were hematopoietic cells contaminating the cell aggregates. Therefore, these cells were excluded from further study, and sorting of CD49f-positive cells and Thy1-positive cells only was performed to characterize these two fractions. The Thy1-positive mesenchymal cell population was further separated into two fractions: a gp38-positive fraction (purity 97%) and a gp38-negative fraction (purity 94%) (FIG. 2H).

Cell Sorting and Immunocytochemistry

CD49f-positive cells and Thy1-positive cells were sorted using FACSVantage, cultured in vitro, and subjected to subsequent immunocytochemical analysis. At day 1 after sorting, CD49f-positve cells appeared morphologically uniform and cuboidal in shape. In addition, these cells possessed large nuclei and highly granulated cytoplasm. Upon immunocytochemical staining, CD49f-positive cells were homogeneously stained for AFP, heterogeneously stained for ALB and CK19, but did not stain for desmin or α-SMA. ALB was expressed more strongly toward the inside of the colony, whereas CK19 was expressed more strongly toward the periphery of the colony. Some double-positive cells were detected. These results were similar to those of a previous report [15].

Thus, immunocytochemical staining of CD49f-positive cells revealed a homogeneous distribution of AFP and heterogeneous patterns of ALB and CK19 staining.

In view of previous reports regarding AFP expression in endodermal cells [16], and c-Met⁺CD49f^{+(low)} c-Kit⁻CD45⁻TER119⁻ cells [17-19], it is likely that the CD49f-positive cells were AFP-producing primitive hepatic endodermal cells and that the heterogeneous staining pattern of ALB and CK19 reflect different populations of hepatic stem/progenitor cells at various stages of differentiation present among the CD49f-positive cells. The antigenic profile of the primitive hepatic endodermal cells we isolated was CD49f⁺ c-Met⁺ c-Kit⁻ CD45⁻, which is consistent with previous reports by Suzuki et al [17-19].

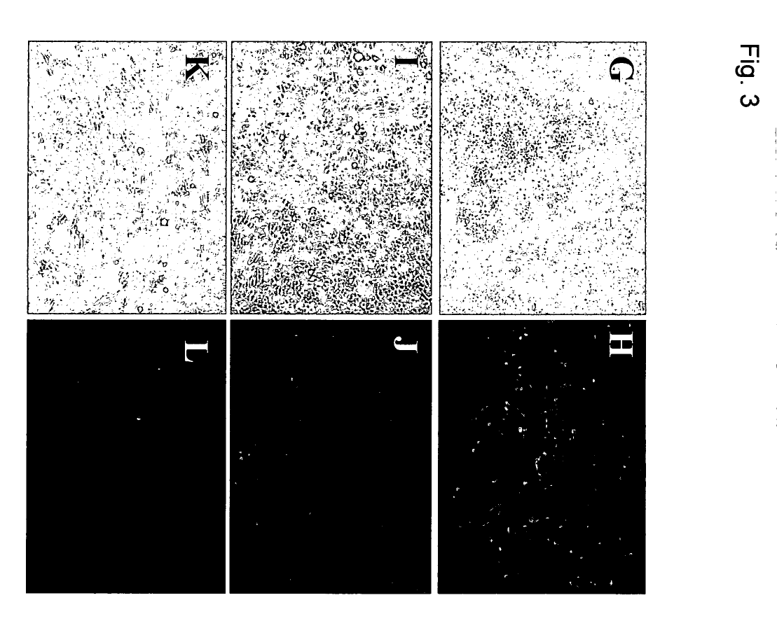

At day 1 after sorting, the Thy1-positive cell population appeared morphologically to comprise two distinct cell types. One was spindle-like in shape and possessed small nuclei. The other had more highly granulated cytoplasm (FIG. 3A). Upon immunocytochemical staining, both cell types stained for α-SMA (FIG. 3B), but only the latter cell type stained for desmin (FIG. 3C). At day 5, round cells with large nuclei appeared in colonies of the first cell type and proliferated (FIG. 3D). This cell type did not stain for either α-SMA or for desmin (FIGS. 3E and 3F). None of these cell types incorporated acetylated low-density lipoprotein or latex microspheres, and none exhibiting AFP, ALB or CK19 staining.

Thus, upon close morphological examination, the population of Thy1-positive cells was found to contain at least three sub-populations. Two of these sub-types stained positive for α-SMA and partially positive for desmin, whereas the remaining cell type did not exhibit either α-SMA or desmin staining. However, none of the cell types stained positive for hepatic endodermal specific markers such as AFP, ALB, and CK19. Furthermore, Thy1-positive cells did not have the characteristics of endothelial cells or Kupffer cells by RT-PCR or FACS analysis.

Desmin is one of the principal intermediate filament proteins expressed in cardiac, skeletal, and smooth muscle cells [20]. In the liver, desmin is selectively expressed in hepatic stellate cells (HSCs) [21, 22]. However, the Thy1-positive cells in the present study were morphologically different from adult HSCs, which do not express the Thy1 surface antigen (data not shown). On the other hand, they are positive for α-SMA, one of six isoactins expressed in mammalian cells and the isoform that is typical of smooth muscle cells [23], especially those in blood vessels [24]. In fibrotic liver and in vitro culture, HSCs change their normal quiescent phenotype to an activated myofibroblast-like phenotype and express α-SMA [25, 26]. Charbord et al. have reported that stromal cells from different developmental sites including bone marrow and fetal liver followed a vascular smooth muscle cell differentiation pathway [27, 28]. Thus, the desmin and α-SMA findings suggest that these Thy1-positive cells are of the mesenchymal lineage and comprise a heterogeneous set of cell populations.

Immunocytochemical Analysis of gp38 in Thy1-Positive Mesenchymal Cells

FIGS. 3G-L show phase contrast (G) and the corresponding fluorescent microscopic (H) images of isolated Thy1-positive cells derived from fetal liver nine days after isolation. The Thy1-positive population contained gp38-positive (red) and -negative cells. Phase contrast (I, K) and fluorescent microscopic (J, L) images of the isolated gp38-positive (I, J) and -negative (K, L) Thy1-positive cells. Original magnifications: G and H, ×100; I-L, ×200. These mesenchymal cell preparations contain two populations, one of a cuboidal shape (I, J) and the other spindle shaped (K, L) in morphology. In this study, we determined that the mucin-type transmembrane glycoprotein 38 (gp38) could distinguish the cuboidal cells (CD49f^{±}Thy1⁺gp38⁺CD45⁻ cells) from the spindle cells (CD49f^{±}Thy1⁺gp38⁻CD45⁻ cells) (FIG. 3I-L).

Further Characterization of Thy1-Positive Cells

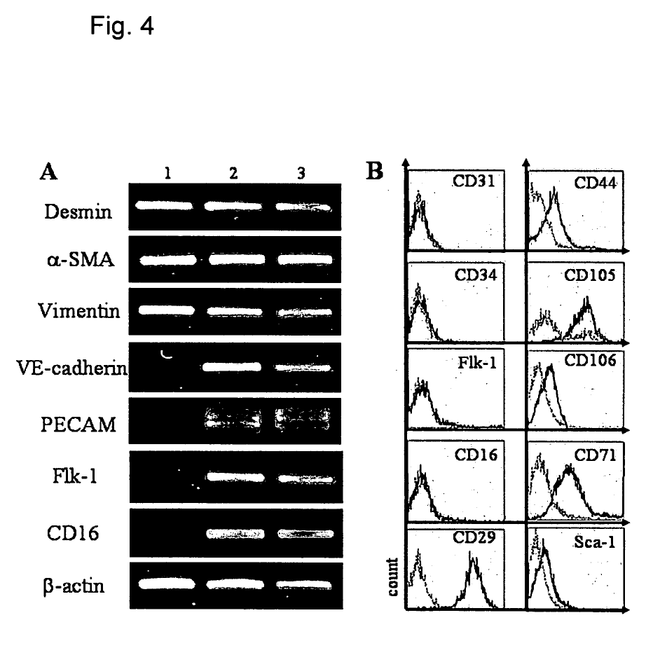

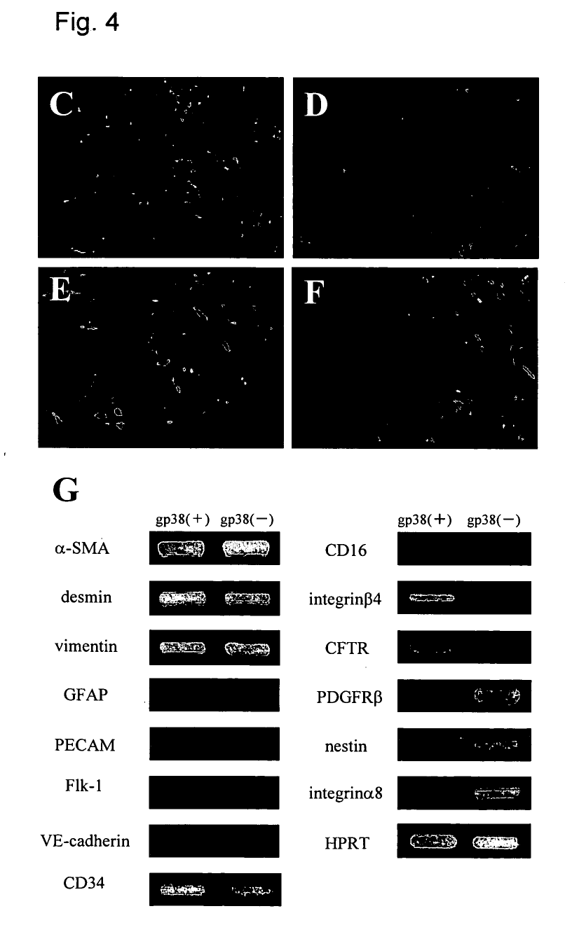

We further examined the Thy1-positive cells using RT-PCR and FACS analysis. RT-PCR demonstrated that the Thy1-positive cells expressed desmin, α-SMA, and vimentin mRNA, but did not express the endothelial cell and Kupffer cell markers VE-cadherin, PECAM, Flk-1, and CD16 (FIG. 4A). Supporting the RT-PCR data, FACS analysis showed that Thy1-positive cells did not express the endothelial cell and Kupffer cell markers CD31, CD34, Flk1, and CD16. Thy1-positive cells were CD29⁺, CD44⁺, CD105⁺, CD106^{±}, CD71^{±}, and Sca-1^{±} (FIG. 4B). Further fractionation of Thy1-positive cells by surface antigen expression was difficult.

These results indicates that the Thy1-positive cells are not hepatic, endothelial, or Kupffer cells, but are mesenchymal cells.

Expression of Mesenchymal Markers by the Two Mesenchymal Cell Populations

Immunocytochemical analysis of gp38-positive (FIG. 4C, E) and -negative (FIG. 4D, F) cells for α-SMA (FIG. 4C, D) and desmin (FIG. 4E, F) was performed. RT-PCR analysis of gp38-positive (left) and -negative (right) cells for α-SMA, desmin, vimentin, GFAP, PECAM, Flk-1, VE-cadherin, CD34, CD16, Integrin β4, CFTR, PDGFR-β, nestin, integrin α8, and HPRT was performed (FIG. 4G). mRNA expression analysis revealed the differences between the isolated CD49f^{±}Thy1⁺gp38⁺CD45⁻ (gp38-positive) cells and CD49f^{±}Thy1⁺gp38⁻CD45⁻ (gp38-negative) cells, although both cells expressed mesenchymal markers.

Morphological and Functional Analyses of the Interaction Between CD49f-Positive Cells and Thy1-Positive Cells

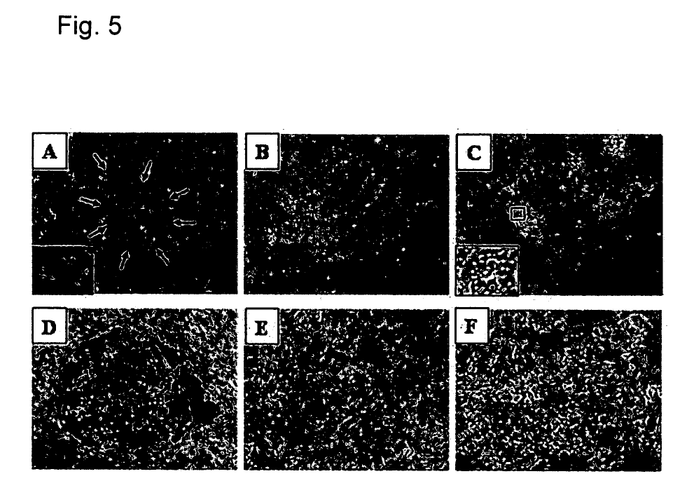

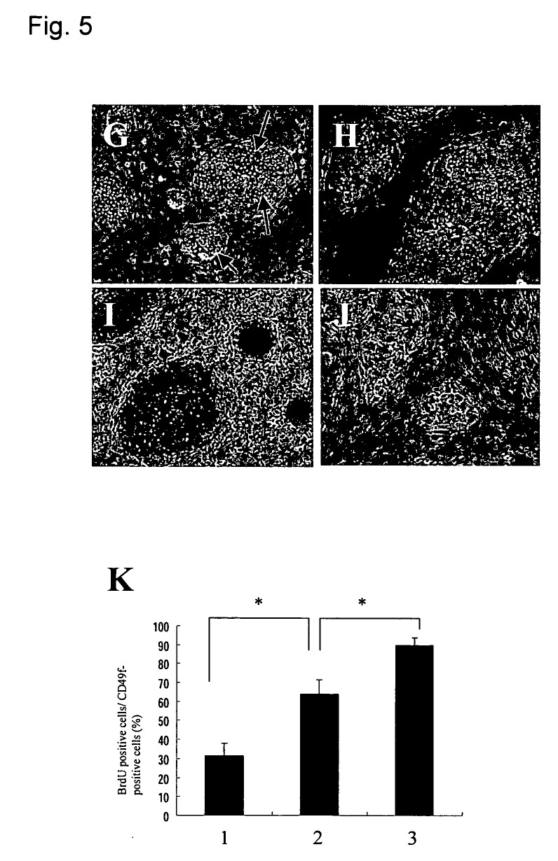

To examine the interaction between CD49f-positive cells and Thy1-positive cells, we co-cultured both cell fractions. When CD49f-positive cells were co-cultured with Thy1-positive cells (method 1), AFP-producing CD49f-positive cells had increased intracellular granularity at day 3 (FIG. 5A), proliferated until day 7, and then piled up from the periphery of the colonies where they were in contact with Thy1-positive cells (FIG. 5B). At day 14, the piled-up area was widely expanded in CD49f-positive colonies and some binucleated cells were detected in the piled-up area (FIG. 5C). In contrast, when CD49f-positive cells were cultured alone or were cultured together with Thy1-positive cells, but without any direct contact (method 2), the CD49f-positive cells in the periphery of the colonies did not maintain their morphological appearance and began to decrease in number at day 3, failing to show any signs of maturation. Supporting these morphological findings, a number of cells in the CD49f-positive colonies were positive for PAS staining at day 10, when CD49f-positive cells were co-cultured with Thy1-positive cells (FIG. 5D). However, CD49f-positive cells cultured alone or separately with Thy1-positive cells were negative for PAS staining even at day 10 (FIGS. 5E and 5F). In our functional analysis, CD49f-positive cells cocultured with gp38-positive cells were positive for Periodic Acid Schiff (PAS) staining, while the gp38-negative cells were negative (FIG. 5G-J). In contrast, the upregulation of BrdU incorporation by CD49f-positive cells revealed the proliferative effect of coculture with gp38-negative cells (FIG. 5K).

These results suggest that in vitro maturation of hepatic progenitor cells promoted by gp38-positive cells may be opposed by an inhibitory effect of gp38-negative cells, which likely maintain the immature, proliferative state of CD49f-positive cells.

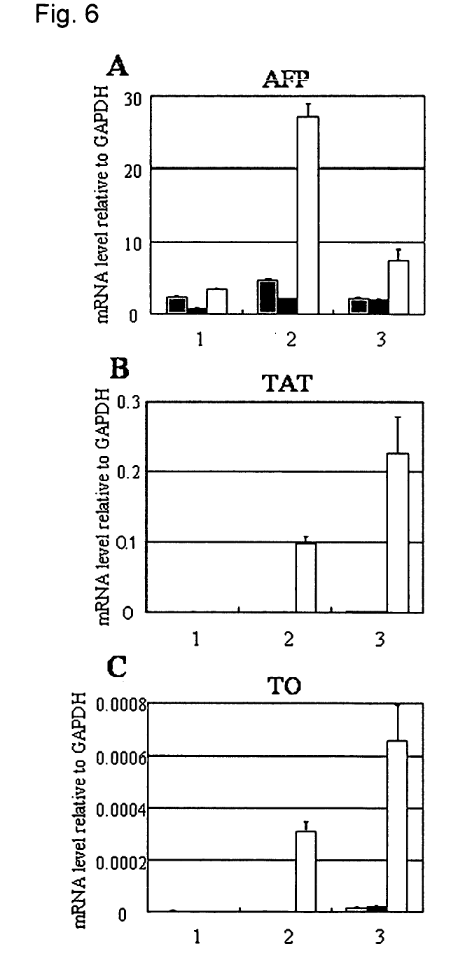

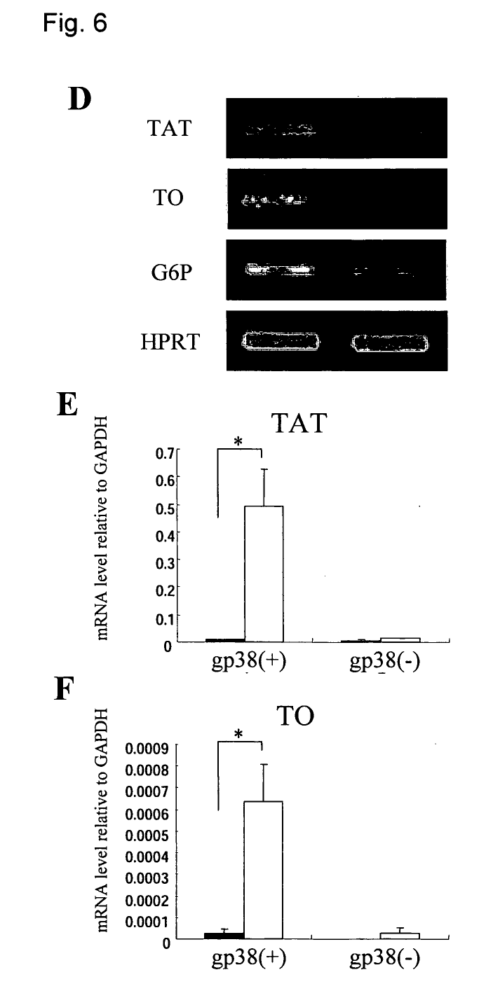

Additionally, in co-cultured cells, the level of TAT and TO mRNA expression was significantly increased (FIGS. 6B and 6C), whereas AFP mRNA was decreased, as assessed by real-time RT-PCR (FIG. 6A). In contrast, no significant increase in TAT and TO mRNA expression was observed in cultures of CD49f-positive cells either alone or in separate cultures together with Thy1-positive cells (FIGS. 6B and 6C). Expression of mature hepatocyte markers, such as tyrosine amino transferase (TAT), tryptophan-2,3-dioxygenase (TO), and glucose-6-phosphatase (G6P), were only upregulated on hepatic progenitors following coculture with gp38-positive cells (FIG. 6D-F).

Transmission Electron Microscopy

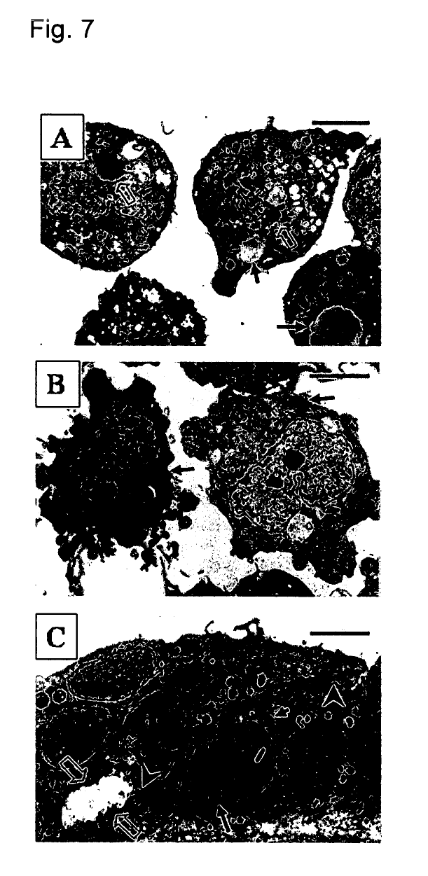

To confirm further the putative maturation of the co-cultured CD49f-positive and Thy1-positive cells, we used transmission electron microscopy to compare the ultrastructures of the cells just after their separation and after 30 days of co-culture. The CD49f-positive cells just after separation had large nuclei with developed nucleoli and a number of fat droplets, but had few intra-cytoplasmic organelles such as mitochondria and peroxisomes (FIG. 7A). The Thy1-positive cells just after separation appeared to comprise two cell types distinguished by their having either clear or dark cytoplasm. This difference was thought to be due to the number of intra-cytoplasmic ribosomes. Both cell types had large nuclei, similar to CD49f-positive cells, a large amount of open rough endoplasmic reticulum, and many microfilaments in the cytoplasm (FIG. 7B). In contrast, the co-cultured cells had no fat droplets, contained many mitochondria, peroxisomes and tight junctions with desmosomes, and formed biliary canaliculi with microvilli (FIG. 7C). All of these features are compatible with those of mature hepatocytes.

Thus, cell-cell contact with Thy1-positive cells was essential for the maturation of primitive hepatic endodermal cells. Therefore, similar to its activity in the hematopoietic system, the Thy1 protein may play an important role in allowing Thy1-positive cells to recognize, adhere to, and promote the maturation of primitive hepatic endodermal cells in the fetal liver. The pathway by which Thy1 must signal this maturation is not yet known, but probably is mediated via a surface antigen on primitive hepatic endodermal cells.

Colonies of AFP-producing CD49f-positive cells subjected to co-culture with Thy1-positive cells morphologically resembled mature hepatocytes; a number of cells contained in these colonies even stored a significant amount of glycogen. Additionally, real-time RT-PCR analysis showed that the co-cultured CD49f-positive and Thy1-positive cells expressed increasing amounts of TAT and TO mRNA over time, and transmission electron microscopy confirmed that they had differentiated into mature hepatocytes by day 30. On the other hand, the Thy1-positive cells did not morphologically resemble endodermal cells and did not express endodermal cell markers.

These results suggest that CD49f-positive cells, but not Thy1-positive cells, are responsible for the expression of TO and TAT observed in the co-culture. Therefore, it is likely that the CD49f-positive cells are primitive hepatic endodermal cells with the capacity to differentiate into mature hepatocytes. In contrast, CD49f-positive cells cultured alone or in separated cultures with Thy1-positive cells failed to exhibit signs of morphological or functional maturation. These results suggest that cell-cell contact with Thy1-positive cells is essential for the maturation of CD49f-positive cells in vitro.

### Example 2

Materials and Methods

Construction of Transgene Vector

The AFP promoter sequence, encompassing nucleotides −794 to +124 of the mouse AFP gene (the adenine of the ATG start codon was numbered as nucleotide 1) was obtained by long-range polymerase chain reaction (PCR) using LA-Taq polymerase (Takara Bio Inc., Otsu, Japan). A fusion gene of the hygromycin resistance with enhanced green fluorescent protein (Hyg/EGFP) was isolated from the pHygEGFP vector (BD Biosciences Clontech, Palo Alto, Calif.) by digestion with BamHI-NotI (Takara Bio Inc.) and ligated to an SV 40-driven neomycin resistance gene derived from the pEGFP-1 vector (BD Biosciences Clontech). This promoterless Hyg/EGFP vector was digested with SacI-SacII (Takara Bio Inc.) and ligated to the AFP promoter region described above, resulting in a construct in which the Hyg/EGFP fusion proteins were expressed under the control of the AFP promoter.

Generation of Transgenic ES Cells

Transgene vectors were transfected by electroporation into mouse ES cells derived from C57BL/6 mice (the kind gift of Dr. T. Tada, Kyoto University, Kyoto, Japan) using a Gene Pulser II (Bio-Rad, Hercules, Calif.) at 500 μF and 500V. Stably transfected cells were selected in the presence of 200 μg/ml G418 (Sigma, St Louis, Mo.) in the presence of a G418-resistant mouse embryonic fibroblast feeder layer. Proper transgene insertion was confirmed by PCR.

ES Cell Growth and Differentiation into Endoderm

Undifferentiated mouse ES cells were cultured in Dulbecco's modified essential medium (DMEM) (Sigma) supplemented with 20% fetal bovine serum (FBS) (HyClone, Logan, Utah), 0.1 mM 2-mercaptoethanol (Sigma), nonessential amino acids (Sigma), 1 mM sodium pyruvate (Sigma), and 1000 U/ml leukemia inhibitory factor (LIF) (ESGRO, Chemicon International Inc., Temecula, Calif.) on a mouse embryonic fibroblast feeder layer treated with mitomycin C (Wako Pure Chemical Co., Osaka. Japan). To induce edodermal cell differentiation, ES cells were transferred to serum- and feeder layer-free culture conditions. Following dissociation, ES cells were replated at a concentration of 2×10⁴ cells/cm² on 60 mm culture dishes coated with collagen type I (BD Biosciences Discovery Labware, Bedford, Mass.) in DMEM supplemented with 10% Knockout SR (Gibco, Grand Island, N.Y.), 2 mM L-glutamine (Sigma), 1 mM sodium pyruvate, penicillin/streptomycin (Gibco), and 200 μg/ml G418 to deplete the feeder layer cells. To evaluate the effects of growth factors, ES cells were divided into three groups: group 1 received 10 μmol/l all-trans retinoic acid (Sigma) and 1000 U/ml LIF; group 2 was given 20 ng/ml basic fibroblast growth factor (bFGF) (Upstage, Lake Placid, N.Y.) and 20 ng/ml of the deleted form of hepatocyte growth factor (dHGF) [50-51] (kindly provided by Snow Brand Product Co., Osaka, Japan); group 3 was administered 1000 U/ml LIF and 10 μg/ml all-trans retinoic acid for the first two days (day 0-day 1), given 20 ng/ml bFGF and 20 ng/ml dHGF for the next five days (day 2-day 6), and treated with 10 ng/ml oncostatin M (R&D System, Inc., Minneapolis, Minn.) for the last three days (day 7-day 9) to the culture media. GFP expression was detected using a fluorescence microscope (IX70; Olympus, Tokyo, Japan).

Flow Cytometry and Cell Sorting

Differentiated ES cells were dissociated in a trypsin (Gibco)-ethylenediaminetetraacetic acid (Dojindo laboratories, Kumamoto, Japan) solution, and then resuspended in 3% FBS-phosphate buffered saline (PBS). Cells derived from mouse fetal liver were prepared as described in EXAMPLE 1 (also see reference [52]). We used the following antibodies for the isolation of Thy1-positive cells from mouse fetal liver: anti-Thy1-fluorescein isothiocyanate (FITC) (Immunotech, Marseille, France), anti-CD49f-phycoerythrin (PE), and anti-CD45-PE (BD Biosciences Pharmingen, San Diego, Calif.). Cells were analyzed on a FACSCalibur flow cytometer (BD Biosciences Immunocytometry Systems, San Jose, Calif.) and isolated using a FACSVantage SE cell sorter (BD Biosciences Immunocytometry Systems).

Cell Culture of GFP-Positive Cells with Thy1-Positive Mesenchymal Cells

Thy1-positive mesenchymal cells from mouse fetal liver were isolated as described in EXAMPLE 1. For use as a feeder cell layer, Thy1-positive cells were grown on collagen type I-coated dishes to approximately 80% confluency, and then treated with 10 μg/ml mitomycin C for 2 hours. On day 7, GFP-positive cells were isolated from the differentiated ES cells by cell sorting on a FACSVantage SE sorter. To evaluate the effect of Thy1-positive fetal liver cells on ES cell-derived endodermal cells, GFP-positive cells were cultured on collagen type I-coated culture dishes or a feeder layer of Thy1-positive fetal liver cells at concentrations of 2.5×10⁴ cells/cm² in DMEM with 10% FBS, 1 mM sodium pyruvate, penicillin/streptomycin, 10 mM nicotinamide (Sigma), 2 mM L-ascorbic acid phosphate (Wako Pure Chemical), insulin-transferrin-selenium supplement (Gibco), 1×10⁻⁷ M dexamethasone (Sigma), 20 ng/ml dHGF, and 10 ng/ml oncostatin M.

Cytological and Immunocytological Analysis

After washing twice in PBS, cultured cells were fixed in 4% paraformaldehyde (Nacalai Tesque, Inc., Kyoto, Japan) for 15 minutes at 4° C., followed by 15 minutes at room temperature. Immunostaining for AFP and albumin was performed as described in EXAMPLE I (also see reference [52]). Prior to immunostaining for Foxa2, nonspecific binding was blocked with 0.4% bovine serum albumin (Sigma) dissolved in 0.1% saponin (Wako Pure Chemical) in PBS. Cells were then incubated with an anti-Foxa2 antibody (Santa Cruz Biotechnology Inc., Santa Cruz, Calif.) diluted at 1:200 for 16 hours at 4° C. Following extensive washing, stained cells were incubated with Cy3-conjugated anti-goat IgG (Sigma) diluted at 1:500 for 30 minutes at room temperature. After staining with secondary antibody, cells were washed and covered with VECTASHIELD mounting medium with DAPI (Vector Laboratories, Inc., Burlingame, Calif.). Periodic acid-Shiff(PAS) staining detected intracellular glycogen, according to the standard protocol described in EXAMPLE 1.

Reverse-Transcription Polymerase Chain Reaction (RT-PCR)

Total RNA was extracted using an RNeasy Mini kit (Qiagen, Chatsworth, Calif.) and treated with RNase-free DNase (Qiagen). Total RNA (2 μg) was reverse-transcribed into cDNA with oligo (dT) 12-18 primer (Invitrogen, Carlsbad, Calif.) using an Omniscript RT kit (Qiagen). PCR utilized Ex Taq polymerase (Takara Bio Inc.) according to the manufacture's instructions. Primers were generated for the following mouse genes (oligonucleotide sequences are given in brackets, followed by the annealing temperature and the number of cycles used for the PCR): GFP (5′-AAGCAGCACGACTTCTTCAA, 5′-CGGCCATGATATAGACGTTG, 60° C., 25 cycles), AFP (5′-TGCTGCAAATTACCCATGAT, 5′-AAGGTTGGGGTGAGTTCTTG, 58° C., 30 cycles), Foxa2 (5′-AGTGGATCATGGACCTCTTCC, 5′-CTTCCTTCAGTGCCAGTTGC, 58° C., 30 cycles), tyrosine amino transferase (TAT) (5′-TCCAGGAGTTCTGTGAACAGC, 5′-AGTATATGGTGCCTGCCTGC, 58° C., 30 cycles), tryptophan 2,3-dioxygenase (TO) (5′-GCTCAAGGTGATAGCTCGGA, 5′-GGAACTCTGCCATCTGTTCC, 58° C., 30 cycles), glucose-6-phosphatase (G6P) (5′-TGCATTCCTGTATGGTAGTGG, 5′-GAATGAGAGCTCTTGGCTGG, 58° C., 30 cycles) and glyceraldehyde-3-phosphate dehydrogenase (GAPDH) (5′-ATTCAAGGGCACAGTCAAGG, 5′-ATCATAAACATGGGGGCATC, 60° C., 25 cycles).

Transmission Electron Microscopy

Isolated GFP-positive cells were cultured on either collagen type I dishes or a feeder layer of Thy1-positive cells for 30 days. Cells were then fixed in 2% glutaraldehyde (Wako Pure Chemical)-PBS for 1 hour and postfixed in 2% osmium tetroxide (Wako Pure Chemical). After embedding the samples in Epon-812 resin (TAAB, UK), ultra-thin sections were cut on an ultramicrotome and stained with uranyl acetate. Sections were examined by transmission electron microscopy (H-7000; Hitachi, Tokyo, Japan).

Results

Differentiation of ES Cells to AFP-Producing Cells

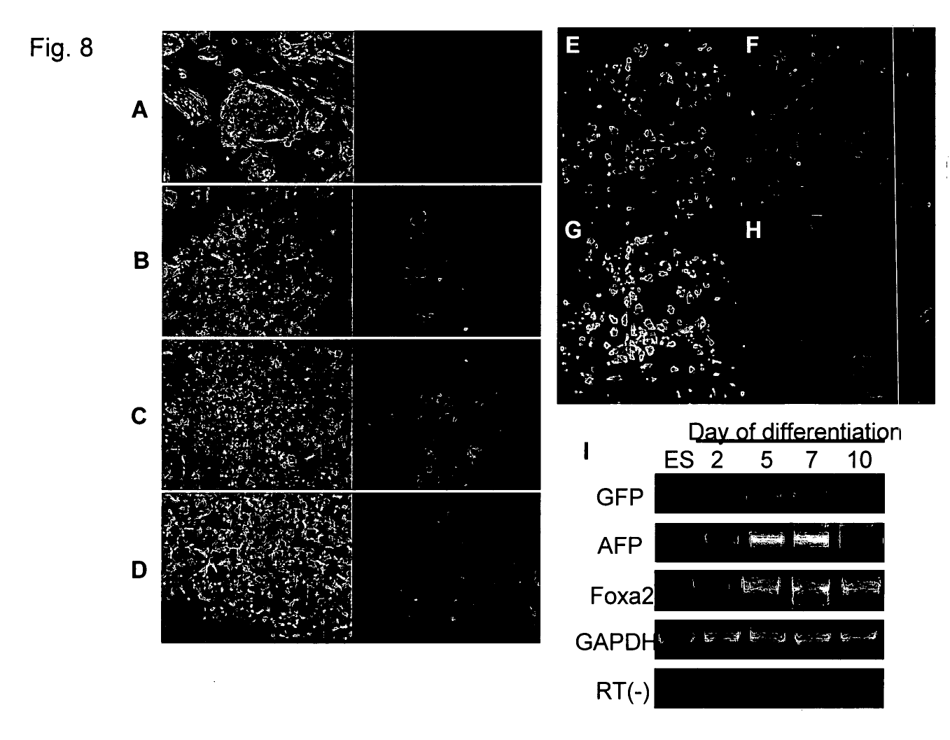

ES cells were transfected with the neomycin resistance construct in which the Hyg/EGFP fusion protein was expressed under the control of the AFP promoter. Eighteen clones of stable transfectants were obtained by G418 selection for 10 days. In comparison to wild-type ES cells, these cells grew normally in the presence of LIF in the presence of a mouse embryonic fibroblast cell feeder layer. Alkaline phosphatase staining and immunostaining for Oct-¾ demonstrated that these cells remained in an undifferentiated state. To evaluate the specificity of GFP expression in AFP-producing cells, transgenic ES cells were transferred to serum- and feeder layer-free conditions for induction of differentiation into endodermal cells. In one clone, GFP expression was detected approximately three days after the induction of differentiation (FIG. 8A-D). The proportion of GFP-positive cells and the individual expression intensity increased gradually until approximately day 7. At day 8, immunostaining for AFP revealed that GFP was coexpressed in AFP-positive cells (FIG. 8E-H). RT-PCR demonstrated that GFP expression was synchronized with AFP (FIG. 8I). Thus, we obtained one transgenic ES cell clone expressing GFP under the control of the AFP promoter. This clone was used for the subsequent experiments.

Flow Cytometric Fractionation of Differentiated ES Cells and Cell Sorting

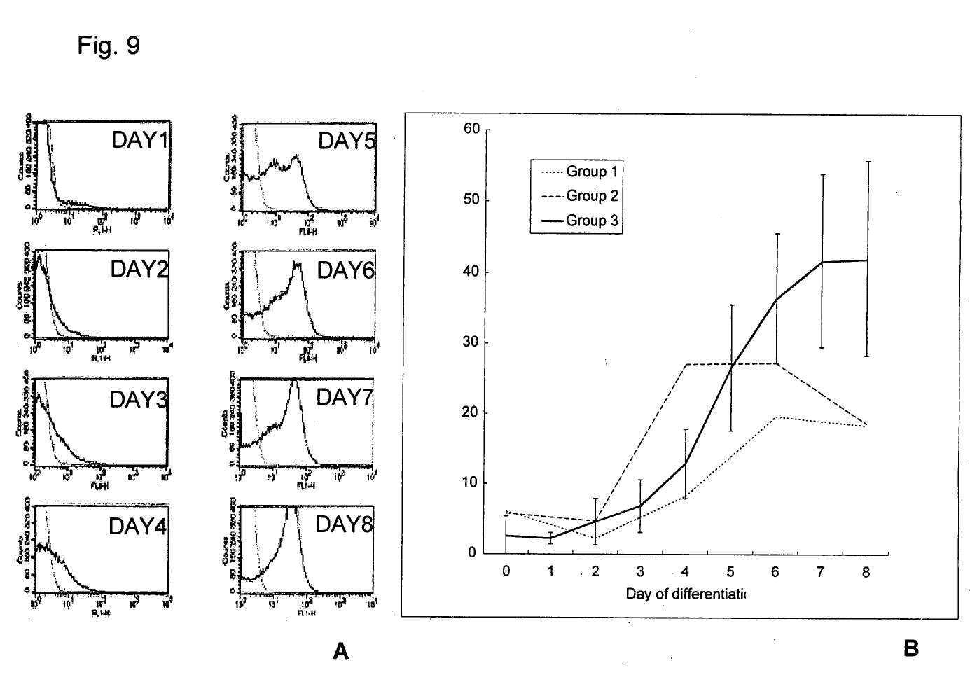

To evaluate the efficiency of GFP-positive cell induction, we performed flow cytometric analyses on each day of the differentiation of ES cells (FIG. 9A). In group 3, graphing of the proportion of GFP-positive cells generated a sigmoid curve plateauing at day 7 to approximately 40% (41.6±12.2%, means±standard deviation). In group 1, this value reached a maximum value of 19.6±2.8% on day 6, while group 2 achieved a maximum proportion of 27.1±7.5% at day 6 (FIG. 9B). GFP-positive cells were then sorted out the total population of group 3 on day 7 by cell sorting using a FACSVantage SE.

Thus, we obtained a differentiation efficiency of approximately 40% for GFP-positive cells at day 7 in group 3. This value did not increase with increasing time. In groups 1 and 2, the relative proportion of GFP-positive cells peaked earlier, but the overall values were lower than that seen for group 3 at day 7.

Characterization of GFP-Positive Cells After Cell Sorting

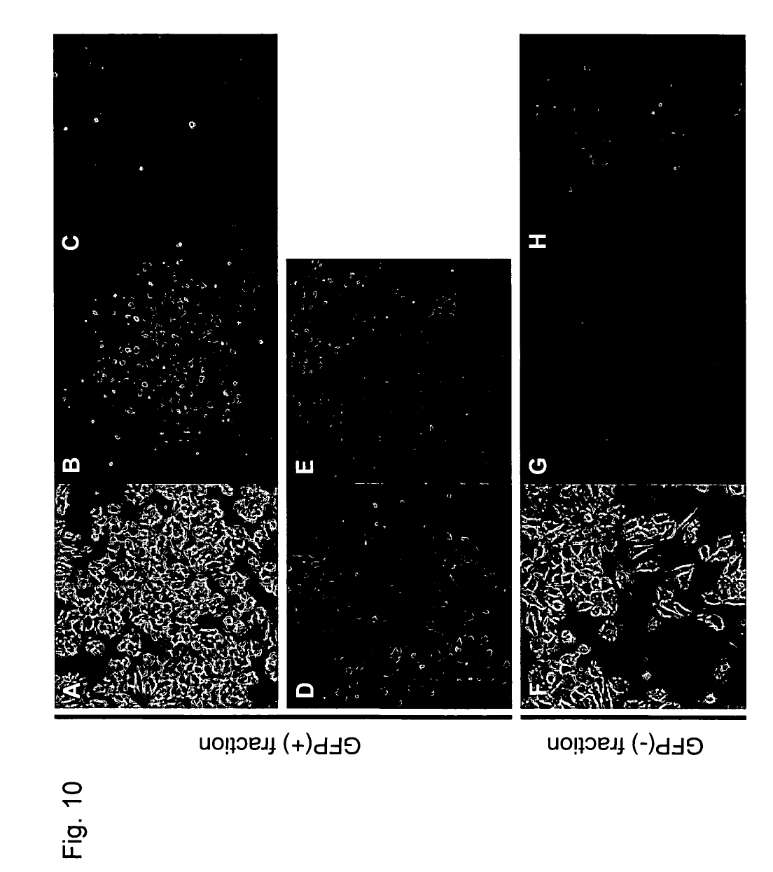

GFP-positive cells appeared morphologically uniform and cuboidal in shape, while GFP-negative cells were comprised of a morphologically heterogeneous cell population. All GFP-positive cells stained for AFP seven days after cell sorting (FIG. 10A-C); in contrast, only a small proportion of the GFP-negative cells stained for AFP (FIG. 10F-H). GFP expression in the GFP-positive fraction was detectable by one week after sorting; this expression was gradually attenuated, disappearing by two weeks. Following culture for 14 days on collagen type I-coated dishes, the vast majority of GFP-positive cells were immunocytologically positive for both albumin and Foxa2 (FIG. 10D, E).

Therefore, GFP-positive cells, considered here to correspond to AFP-producing cells, were thought to include both hepatic progenitor cells and the visceral endoderm of the yolk sac. It is impossible, however, to distinguish between these cell types as there are no markers distinguishing between the definitive endoderm and the yolk sac endoderm at this early stage of development. To define these ES cell-derived endodermal cells as definitive endodermal cells, it is necessary to differentiate ES cells in a manner in accordance with the normal physiological developmental processes. Although the definitive growth factors and molecular mechanisms governing hepatocyte differentiation from ES cells have not yet been well defined, retinoic acid is thought to induce mesodermal differentiation [57-59], and bFGF and hepatocyte growth factor induce endodermal differentiation [59-63]. In our protocol for group 3, ES cells were first induced to differentiate into the mesodermal lineage, then differentiated into the endodermal lineage. Considering that definitive endoderm, the origin of hepatic progenitor cells, is derived from the early gastrula organizer (node) of mesodermal cells [64-66], our group 3 protocol likely induces hepatic progenitor cell differentiation from ES cells in a similar manner as occurs during the physiological developmental process.

A small proportion of the isolated GFP-negative cells stained for AFP. An alternative promoter is located in the first intron of the AFP gene [67-68]. In this study, the AFP promoter region used contained only the authentic AFP promoter, excluding this alternate promoter. Thus, these GFP-negative, AFP-positive cells may produce variant forms of AFP under the control of the alternative AFP promoter. The population of isolated GFP-positive cells was almost completely included in that of AFP-producing cells, exhibiting characteristics similar to hepatic progenitor cells. Nearly all of the isolated GFP-positive cells were positive for AFP, Foxa2, and albumin by immunocytochemistry. As determined by PCR, however, these cells cultured alone did not express late stage markers of heptocyte development, including TAT, TO, and G6P. TAT and G6P are produced in the developing liver at the late fetal and neonatal stages [69-70], and TO is synthesized in the mature liver at the terminal stage of differentiation after birth [71]. The isolated AFP-GFP-positive cells cultured alone were also negative for PAS staining. These data suggest that, while these cells are immature endodermal cells, which include a population of hepatic progenitor cells, the isolated cells cannot maturate into hepatocytes alone in vitro.

Coculture of GFP-Positive Cells with Thy1-Positive Mesenchymal Cells

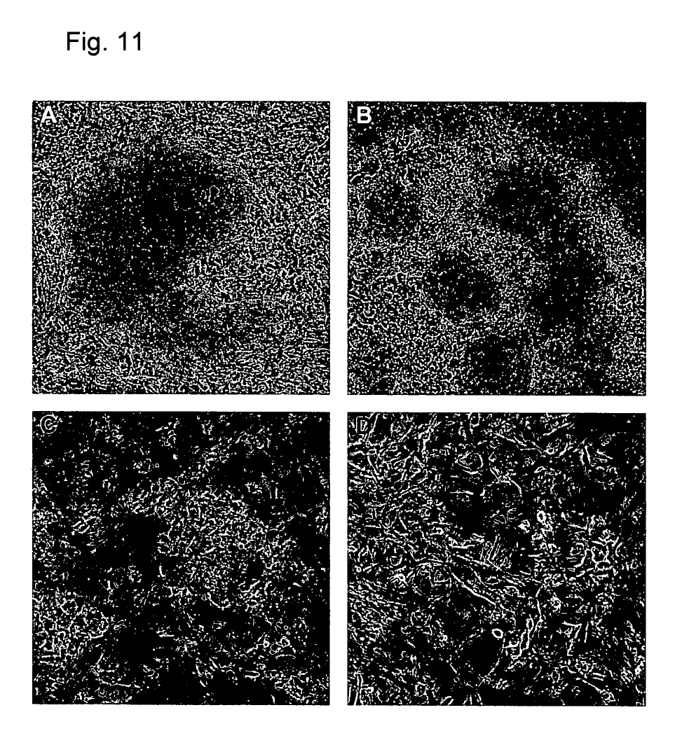

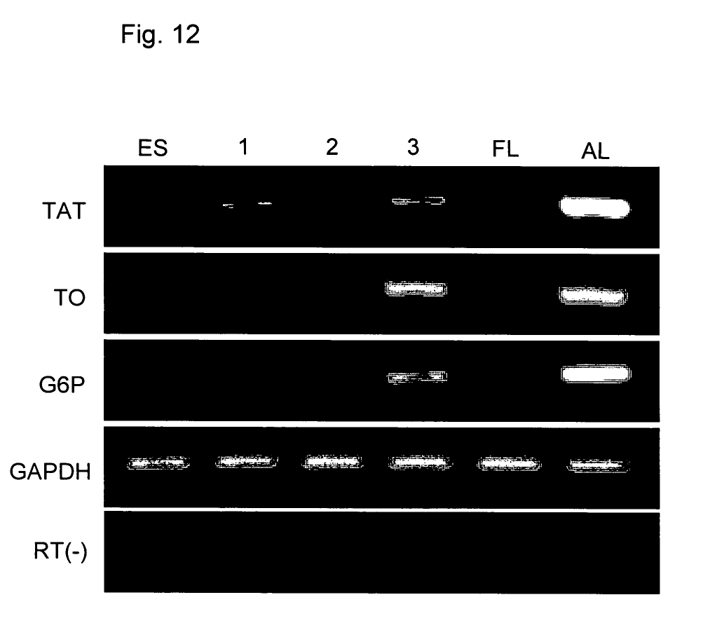

To examine the effect of Thy1-positive fetal liver cells on isolated GFP-positive ES cell-derived cultures, we incubated GFP-positive cells with a feeder layer of Thy1-positive cells. GFP-positive cells proliferated, forming piled-up colonies in seven days of coculture (FIG. 11A). RT-PCR confirmed the expression of mRNAs encoding the mature hepatocyte markers TAT, TO, and G6P in these cells after seven days of coculture (FIG. 12). In contrast, neither Thy1-positive cells nor GFP-positive cells alone expressed these markers, even after culturing for periods greater than one month. We also performed PAS staining to examine glycogen synthesis and storage, one of the functional characteristics of hepatocytes. After seven days of coculture, piled-up regions of cocultures were consistently positive for PAS staining (FIG. 11B), while either Thy1-positive cells or GFP-positive cells cultured alone were negative (FIG. 11C, D).

Thus, Thy1-positive mesenchymal cells promote the maturation of hepatic progenitor cells. Thy1-positive cells were obtained from the fetal livers of mice by flow cytometry. The population of Thy1-positive cells is heterogeneous, including alpha-smooth muscle actin-positive cells. All of the isolated cells, however, are negative for endodermal markers, such as AFP, albumin, and CK19, and do not exhibit the characteristics of either endothelial or Kupffer cells. Therefore, these cells are thought to be cells of the mesenchymal lineage residing in the fetal liver. CD49f-positive hepatic progenitor cells differentiate into mature hepatocytes by direct cell-to-cell contacts with Thy1-positive mesenchymal cells. We hope to apply this coculture system to the differentiation of isolated AFP-GFP-positive endodermal cells into mature hepatocytes.

By RT-PCR analysis, undifferentiated ES cells did not express albumin, TAT, TO, or G6P mRNA, even following coculture with Thy1-positive cells (data not shown). Thus, it is essential to isolate the AFP-producing cell population from the differentiated ES cells prior to coculture with Thy1-positive cells. In cocultures of ES cell-derived AFP-GFP-positive cells with Thy1 positive cells, AFP-GFP-positive cells grew into piled-up colonies, while Thy1-positive cells treated with mitomycin C did not proliferate. These Thy1-positive cells did not resemble endodermal cells morphologically. RT-PCR analysis revealed that AFP-GFP-positive cells cocultured with Thy1-positive cells expressed TAT, TO, and G6P mRNA, while neither AFP-GFP-positive cells nor Thy1-positive cells cultured alone displayed these markers. Analysis of PAS staining demonstrated that the piled-up colonies of AFP-GFP-positive cells produced and stored glycogen only following coculture with Thy1-positive cells.

Transmission Electron Microscopy

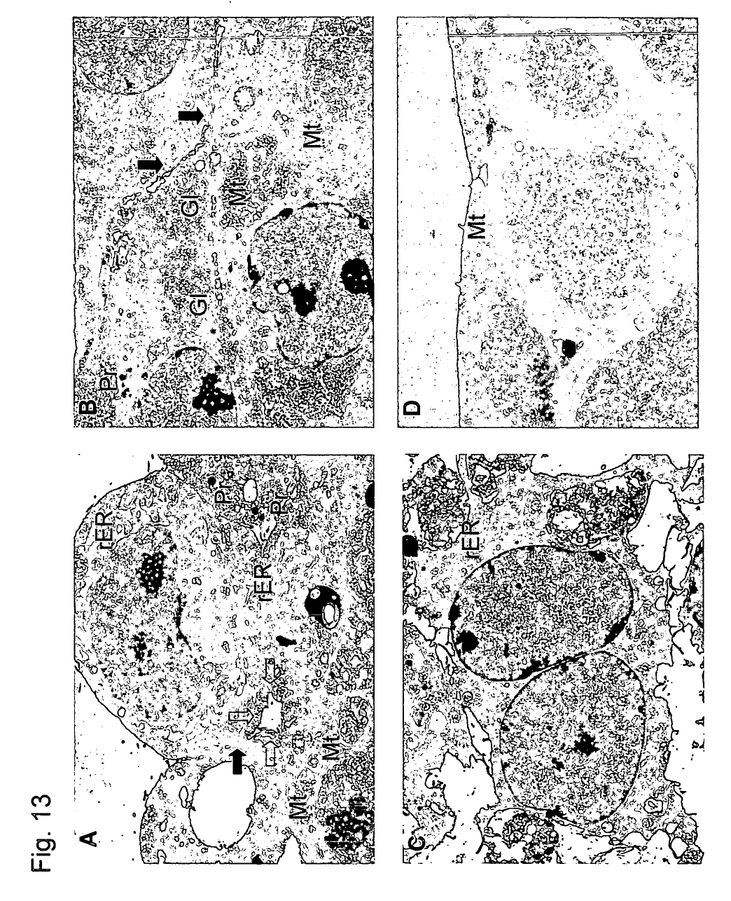

To evaluate the morphological characteristics of the cultured putative hepatocytes, we examined the GFP-positive cells by transmission electron microscopy. Isolated GFP-positive cells were cocultured with Thy1-positive cells for one month. These cells formed piled-up colonies possessing mature hepatocyte-like ultrastructures, such as numerous well-developed mitochondria, high levels of rough endoplasmic reticulum, large numbers of glycogen granules and peroxisomes, and tight junctions with desmosomes (FIG. 13A-C). Furthermore, these cells occasionally formed biliary canaliculi (FIG. 13A); some cells were binucleate (FIG. 13C). In contrast, isolated cells cultured alone retained large nuclei and exhibited few intracytoplasmic organelles, except for occasional mitochondria. Biliary canaliculi were not observed in these cultures (FIG. 13D).

These results suggest that AFP-GFP-positive cells differentiate into mature hepatocyte-like cells in vitro through direct cell-to-cell contacts with Thy 1-positive cells. Consequently, these experiments raise the possibility that the signal transduction occurring in ES cell-derived premature endodermal cells is mediated by the binding of surface molecules expressed on Thy1-positive cells, leading to the upregulation of the expression of genes required for terminal differentiation into hepatocytes.

Ammonia Clearance Activity

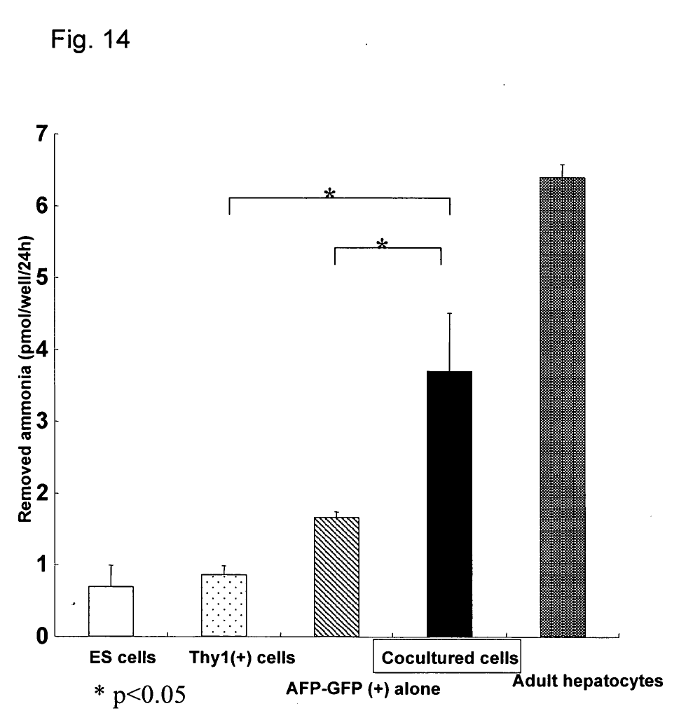

We examined the ammonia metabolism of cultured cells. Ammonia is metabolized into urea by hepatocytes. As shown in FIG. 14, GFP-positive cells cultured alone exhibited low activity to remove ammonia from the culture media. In contrast, cocultured cells displayed an approximately two-fold greater metabolizing activity than that of GFP-positive cells cultured alone (P<0.05). Thy1-positive cells cultured alone did not possess any activity to metabolize ammonia. We evaluated ammonia clearance activity per cell by dividing the amount of removed ammonia by the total number of cells. Cocultured cells metabolized 5.49±1.84 μmol/10⁷ cells/24 h ammonia, while Thy1-positive cells and GFP-positive cells cultured alone removed 1.53±0.03 and 2.41±0.97 μmol/10⁷ cells/24 h ammonia, respectively.

### INDUSTRIAL APPLICABILITY

The present invention is useful for the therapy of liver diseases that require generation of essentially pure endodermal cells and subsequent maturation of the cells into functional hepatocytes in vitro. The present invention is also useful for various bioassays.

### REFERENCES

1. Bilir B M, Guinette D, Karrer F, Kumpe D A, Krysl J, Stephens J, McGavran L, et al. Hepatocyte transplantation in acute liver failure. Liver Transpl 2000;6:32-40.

2. Fox I J, Chowdhury J R, Kaufman S S, Goetzen T C, Chowdhury N R, Warkentin P I, Strom S C, et al. Treatment of the Crigler-Najar syndrome type I with hepatocyte transplantation. N Engl J Med 1998;338:1422-1426.

3. Horslen S P, McCowan T C, Goertzen T C, Warkentin P I, Cai H B, Strom S C, Fox I J. Isolated hepatocyte transplantation in an infant with a severe urea cycle disorder. Pediatrics 2003;111:1262-7.

4. Kobayashi N, Fujiwara T, Westerman K A, Inoue Y, Sakaguchi M, Noguchi H, Leboulch P, et al. Prevention of acute liver failure in rats with reversibly immortalized human hepatocytes. Science 2000;287:1258-1262.

5. Susick R, Moss N, Kubota H, Lecluyse E, Hamilton G, Luntz T, Ludlow J, et al. Hepatic progenitors and strategies for liver cell therapies. Ann New York Acad Sci 2001;944:398-419.

6. Sigal S H, Rajvanshi P, Reid L M, Gupta S. Demonstration of differentiation in hepatocyte progenitor cells using dipeptidyl peptidase IV deficient mutant rats. Cell Mol Biol Res 1995;41:39-47.

7. Sandhu J S, Petkov P M, Dabeva M D, Shafritz D A. Stem cell properties and repopulation of the rat liver by fetal liver epithelial progenitor cells. Am J Pathol 2001;159:1323-34.

8. Zaret K S. Regulatory phases of early liver development: Paradigms of organogenesis. Nat Rev Genet 2002;3:499-512.

9. Miyajima A, Kinoshita T, Tanaka M, Kamiya A, Mukouyama Y, Hara T. Role of oncostatin M in hematopoiesis and liver development. Cytokine Growth Factor Rev 2000; 11:177-83.

10. Kamiya A, Kinoshita T, Miyajima A. Oncostatin M and hepatocyte growth factor induce hepatic maturation via distinct signaling pathways. FEBS Lett 2001 ;492:90-94.

11. Nagai H, Terada K, Watanabe G, Ueno Y, Aiba N, Shibuya T, Kawagoe M, et al. Differentiation of liver epithelial (stem-like) cells into bepatocytes induced by coculture with hepatic stellate cells. Biochem Biophys Res Commun 2002;293:1420-1425.

12. Nitou M, Sugiyama Y, Ishikawa K, Shiojiri N. Purification of fetal mouse hepatoblasts by magnetic beads coated with monoclonal anti-E-cadherin antibodies and their inn vitro culture. Exp cell Res 2002;279:330-343.

13. Koike T, Shiojiri N. Differentiation of the mouse hepatic primordium cultured inn vitro. Differentiation 1996;61:35-43.

14. Bhatia S N, Balis U J, Yarmush M L, Toner M. Effect of cell-cell interactions in preservation of cellular phenotype: cocultivation of hepatocytes and nonparenchymal cells. FASEB J 1999;13:1883-900.

15. Yasuchika K, Hirose T, Fujii H, Oe S, Hasegawa K, Fujikawa T, Azuma H, et al. Establishment of a highly efficient gene transfer system for mouse fetal hepatic progenitor cells. Hepatology 2002;36:1488-1497.

16. Shiojiri N, Lemire J M, Fausto N. Cell lineages and oval cell progenitors in rat liver development. Cancer Res 1991 ;51:2611-2620.

17. Suzuki A, Zheng Y, Kaneko S, Onodera M, Fukao K, Nakauchi H, Taniguchi H. Clonal identification and characterization of self-renewing pluripotent stem cells in the developing liver. J cell Bio 2002; 156:173-184.

18. Suzuki A, Zheng Y, Kondo R, Kusakabe M, Takada Y, Fukao K, Nakauchi H, et al. Flow-cytometric separation and enrichment of hepatic progenitor cells in the developing mouse liver. Hepatology 2000;32:1230-1239.

19. Suzuki A, Iwama A, Miyashita H, Nakauchi H, Taniguchi H. Role for growth factors and extracellular matrix in controlling differentiation of prospectively isolated hepatic stem cells. Development 2003;130:2513-2524.

20. Sappino A P, Schürch W, Gabbiani G Differentiation repertoire of fibroblastic cells: expression of cytoskeletal proteins as marker of phenotypic modulations. Lab Invest 1990;63:144-61.

21. Takase S, Leo M A, Nouchi T, Lieber C S. Desmin distinguishes cultured fat-storing cells from myofibroblasts, smooth muscle cells and fibroblasts in the rat. J Hepatol 1988;6:267-76.

22. Yokoi Y, Namihisa T, Kuroda H, Komatsu I, Miyazaki A, Watanebe S, Usui K. Immunocytochemical detection of desmin in fat-storing cells (Ito cells). Hepatology 1984;4:709-14.

23. Owens G K. Regulation of differentiation of vascular smooth muscle cells. Physiol Rev 1995;75:487-517.

24. Gabbiani GK, Schmid E, Winter S, Chaponnier C, de Ckhastonay C, Vandekerckhove J, Weber K, et al. Vascular smooth muscle cells differ from other smooth muscle cells: predominance of vimentin filaments and a specific a-type actin. Proc Natl Acad Sci U S A 1981;78:298-302.

25. Rockey D C, Boyles J K, Gabbiani G, Friedman S L. Rat hepatic lipocytes express smooth muscle actin upon activation in vivo and in culture. J Submicrosc Cytol Pathol 1992;24: 193-203.

26. Schmitt-Graff A, Kruger S, Bochard F, Gabbiani G, Denk H. Modulation of alpha-smooth muscle actin and desmin expression in perisinusoidal cells of normal and diseased human livers. Am J Pathol 1991;12:1233-1242.

27. Charbord P, Oostendorp R, Pang W, Herault O, Noel F, Tsuji T, Dzierzak E, et al. Comparative study of stromal cell lines derived from embryonic, fetal, and postnatal mouse blood-forming tissues. Exp Hemat 2002;30:1202-1210.

28. Remy-Martin J P, Marandin A, Challier B, Bernard G, Deshaseaux M, Herve P, Wei Y, et al. Vascular smooth muscle differentiation of murine stroma: a sequential model. Exp Hemat 1999;27:1782-1795

29. Craig W, Kay R, Cutler R L, Lansdorp P M. Expression of Thy-1 on human hematopoietic progenitor cells. J Exp Med 1993; 177:1331-1342.

30. Petersen B E, Goff J P, Greenberger J S, Michalopoulos G K. Hepatic oval cells express the hematopoietic stem cell marker Thy-1 in the rat. Hepatology 1998;27:433-445.

31. Walsh F S, Ritter M A. Surface antigen differentiation during human myogenesis in culture. Nature 1981;289:60-64.

32. He H T, Naquet P, Caillol D, Pierres M. Thy-1 supports adhesion of mouse thymocytes to thymic epithelial cells through a Ca²⁺ independent mechanism. J Exp Med 1991;173:515-526.

33. Evans M. J., Kaufman M. H., Establishment in culture of pluripotent cells from mouse embryos, Nature 292 (1981) 154-156.

34. Kawasaki H., Mizuseki K., Nishikawa S., Kaneko S., Kuwana Y., Nakanishi S., Nishikawa S. I., Sasai Y., Induction of midbrain dopaminergic neurons from ES cells by stromal cell-derived inducing activity, Neuron 28 (2000) 31-40.

35. Klug M. G, Soonpaa M. H., Koh G Y., Field L. J., Genetically selected cardiomyocytes from differentiating embryonic stem cells form stable intracardiac grafts, J. Clin. Invest. 98 (1996) 216-224.

36. Lumelsky N., Blondel O., Laeng P., Velasco I., Ravin R., McKay R., Differentiation of embryonic stem cells to insulin-secreting structures similar to pancreatic islets, Science 292 (2001) 1389-1394.

37. Strom S., Fisher R., Hepatocyte transplantation: new possibilities for therapy, Gastroenterology 124 (2003) 568-571.

38. Ohashi K., Park F., Kay M. A., Hepatocyte transplantation: clinical and experimental application, J. Mol. Med. 79 (2001) 617-630.

39. Fox I. J., Transplantation into and inside the liver, Hepatology 36 (2002) 249-251.

40. Hamazaki T., liboshi Y., Oka M., Papst P. J., Meacham A. M., Zon L. I., Terada N., Hepatic maturation in differentiating embryonic stem cells inn vitro, FEBS Lett. 497 (2001) 15-19.

41. Chinzei R., Tanaka Y., Shimizu-Saito K., Hara Y., Kakinuma S., Watanabe M., Teramoto K., Arii S., Takase K., Sato C., Terada N., Teraoka H., Embryoid-body cells derived from a mouse embryonic stem cell line show differentiation into functional hepatocytes, Hepatology 36 (2002) 22-29.

42. Ishizaka S., Shiroi A., Kanda S., Yoshikawa M., Tsujinoue H., Kuriyama S., Hasuma T., Nakatani K., Takahashi K., Development of hepatocytes from ES cells after transfection with the HNF-3beta gene, FASEB J. 16 (2002) 1444-1446.

43. Jones E. A., Tosh D., Wilson D. I., Lindsay S., Forrester L. M., Hepatic differentiation of murine embryonic stem cells, Exp. Cell Res. 272 (2002) 15-22.

44. Kubo A., Shinozaki K., Shannon J. M., Kouskoff V., Kennedy M., Woo S., Fehling H. J., Keller G, Development of definitive endoderm from embryonic stem cells in culture, Development 131 (2004) 1651-1562.

45. Yamamoto H., Quinn G, Asari A., Yamanokuchi H., Teratani T., Terada M., Ochiya T., Differentiation of embryonic stem cells into hepatocytes: biological functions and therapeutic application, Hepatology 37 (2003) 983-993.

46. Yin Y., Lim Y. K., Salto-Tellez M., Ng S. C., Lin C. S., Lim S. K., AFP (+), ESC-derived cells engraft and differentiate into hepatocytes in vivo, Stem Cells 20 (2002) 338-346.

47. Abe K., Niwa H., Iwase K., Takiguchi M., Mori M., Abe S. I., Abe K., Yamamura K. I., Endoderm-specific gene expression in embryonic stem cells differentiated to embryoid bodies, Exp. Cell Res. 229 (1996) 27-34.

48. Spear B. T., Alpha-fetoprotein gene regulation: lessons from transgenic mice, Semin. Cancer Biol. 9 (1999) 109-116.

49. Hoppo T., Fujii H., Hirose T., Yasuchika K., Azuma H., Baba S., Naito M., Machimoto T., Ikai I., Thy1-positive mesenchymal cells promote the maturation of CD49f-positive hepatic progenitor cells in the mouse fetal liver, Hepatology 39 (2004) 1362-1370.

50. Nakmura T., Nawa K., Ichihara A., Partial purification and characterization of hepatocyte growth factor from serum of hepatectomized rats, Biochem. Biophys. Res. Commun. 122 (1984) 1450-1459.

51. Nakamura T., Nishizawa T., Hagiya M., Seki T., Shimonishi M., Sugimura A., Tashiro K., Shimizu S., Molecular cloning and expression of human hepatocyte growth factor, Nature 342 (1989) 440-443.

52. Yasuchika K., Hirose T., Fujii H., Oe S., Hasegawa K., Fujikawa T., Azuma H., Yamaoka Y., Establishment of a highly efficient gene transfer system for mouse fetal hepatic progenitor cells, Hepatology 36 (2002) 1488-1497.

53. Doetschman T. C., Eistetter H., Katz M., Schmidt W., Kemler R., The inn vitro development of blastocyst-derived embryonic stem cell lines: formation of visceral yolk sac, blood islands and myocardium, J. Embryol. Exp. Morphol. 87 (1985) 27-45.

54. Dziadek M. A., Andrews G K., Tissue specificity of alpha-fetoprotein messenger RNA expression during mouse embryogenesis, EMBO J. 2 (1983) 549-554.

55. Shiojiri N., Lemire J. M., Fausto N., Cell lineages and oval cell progenitors in rat liver development, Cancer Res. 51 (1991) 2611-2620.

56. Gualdi R., Bossard P., Zheng M., Hamada Y., Coleman J. R., Zaret K. S., Hepatic specification of the gut endoderm inn vitro: cell signaling and transcriptional control, Genes Dev. 10 (1996) 1670-1682.

57. Osmond M. K., Butler A. J., Voon F. C., Bellairs R., The effects of retinoic acid on heart formation in the early chick embryo, Development 113 (1991) 1405-1417.

58. Ross S. A., McCaffery P. J., Drager U. C., De Luca L. M., Retinoids in embryonal development, Physiol. Rev. 80 (2000) 1021-1054.

59. Schuldiner M., Yanuka O., Itskovitz-Eldor J., Melton D. A., Benvenisty N, Effects of eight growth factors on the differentiation of cells derived from human embryonic stem cells, Proc. Natl. Acad. Sci. USA 97 (2000) 11307-11312.

60. Schmidt C., Bladt F., Goedecke S., Brinkmann V., Zschiesche W., Sharpe M., Gherardi E., Birchmeier C., Scatter factor/hepatocyte growth factor is essential for liver development, Nature 373 (1995) 699-702.

61. Zaret K. S., Hepatocyte differentiation: from the endoderm and beyond, Curr. Opin. Genet. Dev. 11 (2001) 568-574.

62. Deutsch G. Jung J., Zheng M., Lora J., Zaret K. S., A bipotential precursor population for pancreas and liver within the embryonic endoderm, Development 128 (2001) 871-881.

63. Suzuki A., Iwama A., Miyashita H., Nakauchi H., Taniguchi H., Role for growth factors and extracellular matrix in controlling differentiation of prospectively isolated hepatic stem cells, Development 130 (2003) 2513-2524.

64. Lawson K. A., Meneses J. J., Pedersen R. A., Clonal analysis of epiblast fate during germ layer formation in the mouse embryo, Development 113 (1991) 891-911.

65. Hogan B. L. M., Zaret K. S., Development of the endoderm and its tissue derivatives, in: Rossant J., Tam P. P. L., (Ed.), Mouse development, Academic press, San Diego, 2002, pp 301-330.

66. Vincent S. D., Dunn N. R., Hayashi S., Norris D. P., Robertson E. J., Cell fate decisions within the mouse organizer are governed by graded Nodal signals, Genes Dev. 17 (2003) 1646-1662.

67. Scohy S., Gabant P., Szpirer C., Szpirer J., Identification of an enhancer and an alternative promoter in the first intron of the alpha-fetoprotein gene, Nucleic Acids Res. 28 (2000) 3743-3751.

68. Gabant P., Forrester L., Nichols J., Van Reeth T., De Mees C., Pajack B., Watt A., Smitz J., Alexandre H., Szpirer C., Szpirer J., Alpha-fetoprotein, the major fetal serum protein, is not essential for embryonic development but is required for female fertility, Proc. Natl. Acad. Sci. USA 99 (2002) 12865-12870.

69. Kamiya A., Kinoshita T., Ito Y., Matsui T., Morikawa Y., Senba E., Nakashima K., Taga T., Yoshida K., Kishimoto T., Miyajima A., Fetal liver development requires a paracrine action of oncostatin M through the gpl 30 signal transducer, EMBO J. 18 (1999) 2127-2136.

70. Haber B. A., Chin S., Chuang E., Buikhuisen W., Naji A., Taub R., High levels of glucose-6-phosphatase gene and protein expression reflect an adaptive response in proliferating liver and diabetes, J. Clin. Invest. 95 (1995) 832-841.

71. Nagao M., Nakamura T., Ichihara A., Developmental control of gene expression of tryptophan 2,3-dioxygenase in neonatal rat liver, Biochim. Biophys. Acta. 867 (1986) 179-186.

Abbreviations

The following abbreviations are used in the present specification:

hepatic progenitor cell, HPC; hepatic stellate cell, HSC; phosphate buffered saline, PBS; fetal calf serum, FCS; fluorescence-activated cell sorter, FACS; fluorescein isothiocyanate, FITC; phycoerythrin, PE; allophycocyanin, APC; monoclonal antibody, mAb; immunoglobulin G, IgG; α-fetoprotein, AFP; albumin, ALB; cytokeratin 19, CK19; alpha-smooth muscle actin, α-SMA; tyrosine amino transferase, TAT; tryptophan oxygenase, TO; reverse-transcription polymerase chain reaction, RT-PCR; Periodic acid-Schiff, PAS.

What is claimed is:

1. A method for preparing a mature hepatocyte from an embryonic stem cell in vitro, comprising:
(a) culturing the embryonic stem cell so as to differentiate into an endodermal cell;
(b) isolating a population of the endodermal cell from a population of the differenciated cell; and
(c) culturing the isolated endodermal cell in the presence of a Thy1-positive mesenchymal cell.

2. The method according to claim 1, wherein said culturing embryonic stem cell is performed under serum- and feeder layer-free culture conditions.

3. The method according to claim 1, wherein said endodermal cell population comprises a hepatic progenitor cell.

4. The method according to claim 1, wherein said Thy1-positive mesenchymal cell is used as a feeder cell layer.

5. The method according to claim 1, wherein said Thy1-positive mesenchymal cell is gp38-positive.

6. The method according to claim 1, wherein said embryonic stem cell is derived from a mouse.

7. The method according to claim 1, wherein said embryonic stem cell is transfected with a neomycin resistance construct which contains a Hyg/EGFP fusion protein gene under the control of an AFP promoter.

8. The method according to claim 7, wherein said endodermal cell is an AFP-GFP-positive cell.

9. A mature hepatocyte, which is prepared by the method according to claim 1.

10. A method for preparing a CD49f-positive cell and/or a Thy1-positive cell from a fetal hepatic progenitor cell, comprising:
(a) enriching the fetal hepatic progenitor cell through formation of a cell aggregate;
(b) dissociating the cell aggregate into single cells;
(c) labeling the dissociated cell with a labeled antibody including an antibody specific to CD49f and Thy1; and
(d) separating the labeled cell by cell separation means to isolate a CD49f-positive cell and/or a Thy1-positive cell.

11. The method according to claim 10, wherein the step (b) of dissociating the cell aggregate into single cells comprises:
(e) inoculating the cell aggregate on a type I collagen-coated culture plate to form a monolayer colony; and
(f) incubating the cell adhered to the culture plate with a trypsin-EDTA solution.

12. The method according to claim 10, further comprising:
(g) separating the Thy1-positive cells into a gp38-positive and a gp38-negative fractions.

13. The method according to claim 10, wherein said fetal hepatic progenitor cell is obtained from a fetal liver.

14. The method according to claim 10, wherein said labeled antibody is labeled with a fluorescence dye.

15. The method according to claim 10, wherein said cell separation means is a fluorescence-activated cell sorter.

16. A method for preparing a mature hepatocyte in vitro, comprising:
coculturing a CD49f-positive cell with a Thy1-positive cell, wherein said CD49f-positive cell and said Thy1-positive cell are derived from a fetal hepatic progenitor cell.

17. The method according to claim 16, wherein said Thy1-positive cell is gp38-positive.

18. The method according to claim 16, wherein said CD49f-positive cell and said Thy1-positive cell are prepared by the method according to claim 10.

19. A method for treating a liver disease, comprising:
administering the mature hepatocyte according to claim 9 to a recipient.

20. A pharmaceutical composition for treating a liver disease, comprising the mature hepatocyte according to claim 9 and a pharmaceutically acceptable carrier.
